(19) United States
(12) Patent Application Publication
(10) Pub. No.: US 2013/0273074 A1
(43) Pub. Date: Oct. 17, 2013

(54) Methods and Compositions Using Anti-LPS Ligands for the Treatment and Prevention of Inflammatory Disorders

(71) Applicants: Grant Thomas Rawlin, Kilmore East (AU); Zeil Rosenberg, Closter, NJ (US); Oren Fuerst, New York, NY (US)

(72) Inventors: Grant Thomas Rawlin, Kilmore East (AU); Zeil Rosenberg, Closter, NJ (US); Oren Fuerst, New York, NY (US)

(73) Assignee: IMMURON LIMITED, North Melbourne, Victoria (AU)

(21) Appl. No.: 13/877,780

(22) PCT Filed: Oct. 4, 2011
(86) PCT No.: PCT/AU2011/001266

§ 371 Date: Jun. 18, 2013

(30) Foreign Application Priority Data
Oct. 4, 2010 (US) 61389346

(51) Int. Cl. A61K 39/40; A61K 45/06

(52) U.S. Cl. CPC A61K 39/40; A61K 45/06
USPC 424/164.1; 530/387.5

(57) ABSTRACT

The present invention provides methods and compositions useful in the field of medicine, and particularly in the treatment of inflammatory disorders. More particularly, the invention relates to the use of methods and compositions for the treatment and prevention of disorders associated with inflammation of alimentary tract, such as human immunodeficiency virus (HIV) infection and ulcerative colitis and Crohn's disease.

### FIELD

The present invention provides methods and compositions useful in the field of medicine, and particularly in the treatment of inflammatory disorders. More particularly, the invention relates to the use of methods and compositions for the treatment and prevention of disorders associated with inflammation of alimentary tract, such as human immunodeficiency virus (HIV) infection and ulcerative colitis and Crohn's disease.

### BACKGROUND

The alimentary tract is a prone to inflammatory disorders, a fact due at least in part to the presence of associated immune tissue. The gastrointestinal tract is colonized by an abundance of bacteria, which are in constant interaction with the epithelial lining usually leading to an intricate balance between tolerance and immunological response. In certain circumstances exposure of the gut mucosa to foreign antigens (both microbial and non-microbial) may trigger inflammation that may further radiate to affect remote tissues in the body.

It is now recognized that inflammation of the alimentary tract is significant in infection with HIV. More than 36 million people throughout the world are affected by HIV/AIDS, a devastating disease that presents significant challenges in developed and developing countries alike. In developing countries, the disease threatens to reverse decades of development because it attacks individuals in their most productive years, destroys communities, disrupts food production and places a heavy burden on already weak health services. Quite apart from the accepted economic and developmental issues, infection with HIV is the cause of significant human suffering throughout the world.

Complications of the alimentary tract are known to be the result of HIV infection, exclusive of antiviral chemotherapy. Chronic diarrhea is recognized as a hallmark of advanced HIV infection especially in developing countries, and is often caused by infections. In this setting, the spectrum of causes is broad, significant morbidity is typical as is a reduced quality of life, and mortality is high. Among patients with advanced HIV disease (CD4+ counts <50 cells/μl), opportunistic infections are the most common cause of disease, particularly parasites such as Cryptosporidium and Microsporidium. In the developed world, CMV colitis is a significant cause of morbidity in those with advanced immunodeficiency. Mycobacterium avium complex, commonly seen in the pre-highly active antiretroviral therapy (HAART) era, is now less common and is most likely to be found in the patient who first presents with end-stage HIV infection. Mycobacterium tuberculosis can involve the gut and is an important cause of disease in AIDS patients in the developing world.

In addition to diarrhea, HIV patients are commonly afflicted by an enteropathy characteristic of the disease. These pathological changes may be noted in the absence of opportunistic infectious agents in the gut, or the use of anti-retroviral drugs. HIV enteropathy can manifest as diarrhea, increased GI inflammation, increased intestinal permeability, and malabsorption of certain nutrients. There are many theories on the causes of this enteropathy, with no clear mechanism being provided in the prior art. In light of this, there is currently no accepted protocol for the treatment of HIV enteropathy, apart from symptomatic therapies.

Reported evidence suggests that HIV itself may be an indirect diarrheal pathogen because viral proteins have been found in the gut. HIV has been identified in histologic specimens from the GI tract tissue in up to 40% of patients. The virus is confined to lamina propria macrophages and enterochromaffin cells and has not found in epithelial cells. Intestinal HIV infection may also affect local humoral immunity and cause motility disturbances via effects on autonomic nerves.

HIV patients may also exhibit a generalized immune activation, both systemically and in the gut-associated lymphoid tissue (GALT). This can lead to the infiltration of leukocytes, which are swiftly infected and destroy by the virus. The destruction of key immune cells leads to enhanced disease progression and the opportunity for further infection of the patient with opportunistic organisms.

Since the discovery of HIV, significant progress has been made in the treatment of HIV infection by way of anti-retroviral drugs. While new drugs are often efficacious, resistance to a drug often occurs in due course. The use of combination therapies (of two, three or more drugs) has been used to partially overcome resistance leading to improvements in health and life expectancy. While modern chemotherapy regimes can significantly extend the life spans of HIV-infected individuals, improvements are needed in the efficacy of these drugs.

Crohn's disease is a chronic inflammatory disease of the gastrointestinal tract which can affect any part of the gut, from the mouth to the anus, but commonly affects the small and large intestine. It is one of the group of inflammatory bowel diseases (IBD) which may be genetically linked.

Ulcerative colitis is considered a chronic systemic inflammatory disorder, limited to the large intestine. Ulcerative colitis usually causes inflammation of the rectum and then extends to involve various degrees of the colon. The disease may be limited to just a small section of the colon or it may extend to involve the entire colon (pancolitis).

Patients with inflammatory bowel disease are usually treated with an aminosalicylate medication. These medications have been shown to induce remission in mild to moderate active disease and prevent exacerbations. Where the disease is not adequately managed with aminosalicylates, oral steroids may be added to the treatment regimen. Immunosuppressive medications (such as cyclosporine or azathioprine) may also be used in very refractory cases or to reduce the amount of steroid required. The immunosuppression caused by such agents is clearly adverse to the patient.

More recently, biological agents have been trialed against ulcerative colitis. TNF inhibitors such as infliximab and entanercept and adalimumab have been shown to be efficacious. Side effects of these agents include ocular inflammation and drug-induced lupus.

As will be apparent from the foregoing review of the prior art, there remain significant problems to be overcome in the prevention and treatment of inflammatory conditions of the alimentary tract. It is an aspect of the present invention to overcome or ameliorate a problem of the prior art by providing compositions and methods for the prevention and treatment of inflammatory conditions of the alimentary tract.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### BRIEF DESCRIPTION OF THE FIGURES

FIG. 1 shows SDS-PAGE (10% acrylamide, Coomassie stain) of E. Coli flagellin preparations. Lanes as follows:

| 1. | Precision Plus marker (Bio-Rad) | | |
|---|---|---|---|
| 2. | E. coli B7A | O148:H28 | ETEC |
| 3. | E. coli H10407 | O78:H11 | ETEC |
| 4. | E. coli E123-7 | O128:H21 | ETEC |
| 5. | E. coli B2C | O6:H16 | ETEC |
| 6. | E. coli E11881A | O25:H24 | ETEC |
| 7. | E. coli LF82 | O83:H1 | AIEC |
| 8. | E. coli K99 | | Bovine ETEC |
| 9. | E. coli E8772/0 | O153:H12 | ETEC |
| 10. | E. coli HS | H4 | Human isolate |
| 11. | E. coli HB101 | non motile | K12 derivative |

| | |
|---|---|
| 12. | Precision Plus marker (Bio-Rad) |

FIG. 2 shows a Western blot of the gel shown in FIG. 1, as probed by the anti-flagellin antibodies produced in Example 3. Lanes are as for FIG. 1, with the exception that lane 12 is Magic Mark XP (Invitrogen™).

FIG. 3 shows the schedule of events in the Human Clinical Trial performed.

### SUMMARY OF THE INVENTION

In a first aspect the present invention provides a method for treating or suppressing an inflammatory gastrointestinal disorder in a human subject comprising administering to the subject an effective amount of a medicament comprising a polyclonal anti-LPS antibody. Without wishing to be limited by theory, it is proposed that anti-LPS antibody acts to bind a microbe or microbial product thereby inhibiting translocation across the lining of the gastrointestinal tract.

In one embodiment of the composition, the inflammatory gastrointestinal disorder is an inflammatory bowel disease such as ulcerative colitis, Crohn's disease, irritable bowel syndrome or celiac disease. While the aetiologies of these disorders may be multifactorial, all have an inflammatory component. The inflammatory reactions present in these disorders typically originate in the alimentary tract, causing local and sometimes remote damage to tissues.

In one embodiment, the human subject has a HIV infection. In another embodiment, the inflammatory gastrointestinal disorder is HIV mediated inflammatory bowel disorder.

In another embodiment, the human subject is suffering from AIDS.

In one embodiment, the medicament comprises hyperimmune colostrum raised in bovine mammals by immunization of the bovine mammals with LPS.

The anti-LPS antibody may be administered in a dose in the range of from 1.05 to 325 mg per day. In another embodiment, the anti-LPS antibody may be administered in a solid oral unit dosage form comprising in the range from 1.05 to 325 mg polyclonal anti-LPS antibody. The oral solid dose form may comprise at least 20% by weight hyperimmune bovine colostrum wherein solid bovine colostrum comprises at least 7% by dry weight of the powder of IgG

The medicament comprising hyperimmune colostrum may be raised in bovine mammals by immunization of the bovine mammals with LPS from two or more strains of bacteria.

In another embodiment, the anti-LPS antibody may be administered concomitantly with antiretroviral drugs, preferably selected from the group consisting of Zidovudine (AZT), Abacavir, Emtricitabine (FTC), Lamivudine (3TC), Didanosine (ddI), Stavudine (d4T), Zalcitabine (ddC), Nevirapine, Efavirenz, Delavirdine, Tenofovir, Enfuvirtide (T20), Maraviroc (CCR5), Lopinavir, Atazanavir, Fosamprenvir, Amprenavir, Saquinavir, Indinavir, Nelfinavir, Raltegravir, and Elvitegravir.

In another aspect, the present invention provides a medicament for treatment or suppression of inflammatory gastrointestinal disease in a human subject having HIV infection comprising an effective amount of a polyclonal anti-LPS antibody.

In another aspect, the present invention provides the use of a polyclonal anti-LPS antibody in manufacture of a medicament for administration to a human subject for treatment or suppression of HIV mediated inflammatory bowel disease.

In one embodiment, the medicament is a solid oral unit dosage form comprising in the range from 1.05 to 325 mg polyclonal anti-LPS antibody.

In another aspect, the present invention provides a solid oral unit dose form for treatment or suppression of inflammatory bowel disease in a patient suffering from HIV infection, the solid dosage form comprising at least 20% by weight of hyperimmune bovine colostrum powder based on the total weight of oral dosage form, said hyperimmune bovine colostrum powder comprising at least 7% by dry weight of IgG.

In one embodiment the unit dosage form comprises in the range from 1.05 to 325 mg polyclonal anti-LPS antibody.

Throughout the description and the claims of this specification the word “comprise” and variations of the word, such as “comprising” and “comprises” is not intended to exclude other additives, components, integers or steps.

### DETAILED DESCRIPTION OF THE INVENTION

The present invention is predicated at least in part on the proposal that the administration of a ligand capable of binding to a microbe or a microbial product thereof is useful in the treatment and/or prevention of inflammatory disorders. Without wishing to be limited by theory, it is thought that the ligand inhibits the translocation of the microbe or product (whether it is free, or as a part of a bacterium) across the barrier normally presented by the lining of the alimentary tract. Inhibition of the translocation of the microbe or product may subsequently inhibit inflammation of the alimentary tract, or even in remote tissues of the body. It is proposed that local inflammation of the alimentary tract may be problematic leading to various negative clinical outcomes such as colitis, destruction of CD4+ cells, immune activation, and the like. The treatment or prevention of Inflammation at sites remote to the alimentary tract are included within the scope of this invention. Given that alimentary tract is well vascularized, inflammatory mediators such as interleukin-1, tumor necrosis factor, interleukin-6, interleukin-11, interleukin-8/chemokines, eotaxin, interleukin-16, interleukin-17, colony stimulating factors, interleukin-3, interleukin-4, interleukin-5, interleukin-7, interleukin-9, interleukin-10, interleukin-13, interleukin-14, transforming growth factor-b, interleukin-2, interleukin-12, interleukin-15, interferons, and IFN-g-inducing factor are able to reach remote areas of the body to trigger inflammation. In particular reticuloendothelial tissues such as spleen, bone marrow, liver, lymph nodes and the thymus may be affected by inflammatory mediators generated by the alimentary tract.

Accordingly, in a first aspect the present invention provides a method for treating or suppressing an inflammatory gastrointestinal disorder in a human subject comprising administering to the subject an effective amount of a medicament comprising a polyclonal anti-LPS antibody. Without wishing to be limited by theory, it is proposed that anti-LPS antibody acts to bind a microbe or microbial product thereby inhibiting translocation across the lining of the gastrointestinal tract.

As used herein, the term “microbial product” is intended to include any molecule that is naturally secreted or artificially released from a microbe. The product maybe released from a microbe by a physical method (such as shearing, heating, freezing, thawing, pressurizing), a chemical method (such as oxidation, reduction, acid treatment, alkali treatment), or a biological method (such as enzymatic digestion).

In one embodiment of the invention, the microbial product is a lipopolysaccharide (LPS). In another embodiment, the microbial product is not a lipopolysaccharide.

The inflammation that is causative of the inflammatory disorder may be of an acute nature, usually of sudden onset, in which vascular and exudative processes predominate. The inflammation may be chronic inflammation (a prolonged and persistent inflammation marked chiefly by new connective tissue formation), exudative inflammation (where the prominent feature is an exudates), fibrinous inflammation (characterized by an exudate of coagulated fibrin), granulomatous inflammation (a form, usually chronic, marked by granuloma formation), hyperplastic inflammation (leading to the formation of new connective tissue fibers), interstitial inflammation (affecting chiefly the stroma of an organ), parenchymatous inflammation (affecting chiefly the essential tissue elements of an organ), plastic inflammation, productive inflammation, proliferous inflammation, pseudomembranous inflammation (an acute inflammatory response to a powerful necrotizing toxin, with formation, on a mucosal surface, of a false membrane composed of precipitated fibrin, necrotic epithelium, and inflammatory white cells), purulent inflammation, serous inflammation (one producing a serous exudates), subacute inflammation (a condition intermediate between chronic and acute inflammation, exhibiting some of the characteristics of each), suppurative inflammation (marked by pus formation), or ulcerative inflammation (that in which necrosis on or near the surface leads to loss of tissue and creation of a local defect (ulcer)). It will be understood that more than one type of inflammation may be in operation in any inflammatory disorder. As used herein, the term “disorder” is intended to include any negative alteration to a structure or function of the alimentary tract, such that the health or wellbeing of the subject is adversely affected.

In one form of the composition, the inflammatory disorder is a disorder of the alimentary tract. As used herein the term “gastrointestinal” is ised interchangeably with “alimentary” and “includes the entire length of the structure including the lips, mouth, tongue, pharynx, oesophagus, stomach, duodenum, small intestine, cecum, appendix, ascending colon, transverse colon, descending colon, rectum and anus of the subject. Also included are organs or structures that are in physical communication with the lumen of the alimentary tract including the liver, gall bladder, pancreas, and the salivary glands.

Inflammatory disorders of the alimentary tract may involve any one of more of the various tissues, regions, structures or organs of the alimentary tract including the mucosa (mucositis), mouth (stomatitis), tongue (glossitis), gums (gingivitis), oesophagis (oesophagitis), stomach (gastritis), colon (colitis), ilieum (ileitis), liver (hepatitis), gallbladder (cholecystitis), pancreas (pancreatitis), or parotid salivary gland (parotitis). Clinically, the inflammatory disorder of the alimentary tract may present as increased permeability leading to diarrhea, enteropathy, pain, bloating, constipation, anorexia or malabsorption leading to a decline in body weight, failure to rebuild immune memory in the alimentary tract via CCR5+/CD4+ cells, hypergammaglobulinaemia and cachexia.

The inflammatory gastrointestinal disorder may be an inflammatory bowel disease. Inflammatory bowel disease (IBD) is a group of inflammatory conditions of the large intestine and, in some cases, the small intestine. The most prevalent forms of IBD are Crohn's disease, ulcerative colitis. The main difference between Crohn's disease and ulcerative colitis is the location and nature of the inflammatory changes. Crohn's can affect any part of the gastrointestinal tract, from mouth to anus, although a majority of the cases start in the terminal ileum. Ulcerative colitis, in contrast, is restricted to the colon and the rectum.

Microscopically, ulcerative colitis is restricted to the mucosa (epithelial lining of the gut), while Crohn's disease affects the whole bowel wall. Both Crohn's disease and ulcerative colitis present with extra-intestinal manifestations (such as liver problems, arthritis, skin manifestations and eye problems) in different proportions.

The scope of the present invention extends to less prevalent forms of IBD such as collagenous colitis, lymphocytic colitis, ischaemic colitis, diversion colitis, Behçet's syndrome, infective colitis, and indeterminate colitis. Also included is irritable bowel syndrome, a condition that is not normally classified as an IBD. However, it has been suggested that irritable bowel syndrome includes an inflammatory component.

It is proposed that all IBDs will benefit from the present invention given their common inflammatory mechanisms.

Inflammation of the gut can in turn lead to increased intestinal permeability. The intestine is lined with a single layer of epithelial cells. In the small bowel these epithelial cells are called enterocytes. Enterocytes form the intestinal barrier being joined to each other by tight junctions or zonula occludens to form a barrier to fluid and proteins. The tight junctions may open up the spaces between the cells, the paracellular space allowing movement of intestinal contents inside the body. Abnormally leaky tight junctions result in increased intestinal permeability or a “leaky gut”. A breakdown in the barrier normally provided by the tight junctions can allow fluids and electrolytes to migrate into the lumen of the gut, or facilitate the entry of pathological microbes.

The inflammatory disorder for which the present methods are relevant may be caused by, or associated with an infection of the alimentary tract. Inflammatory responses are important in the eradication of foreign antigens from the body, however these responses can be excessive or otherwise cause adverse effects in the subject. The infection may be caused by, or associated with a microbe such as a virus, bacterium, parasite, fungus, bacteria, or mycoplasma.

For example, many viruses are known to cause inflammation in the gut including those that are responsible for gastroenteritis. Relevant viruses include the rotaviruses, noroviruses, adenoviruses, sapoviruses and astroviruses. Also relevant are the hepatitis viruses including types A, B, C, C, delta agent, E and G.

A further virus for which inflammation is particularly problematic is HIV. Thus, in one form of the method the inflammatory disorder is caused by or associated with infection with HIV, and also AIDS in some circumstances. Inflammation of the alimentary tract is often seen in the HIV infected patient, resulting in significant morbidity.

In one embodiment, the human subject has a HIV infection. In another embodiment, the inflammatory gastrointestinal disorder is HIV mediated inflammatory bowel disorder. In another embodiment, the human subject is suffering from AIDS.

As discussed in the Background section herein, many disorders of the gastrointestinal/alimentary tract are noted clinically in the HIV-infected patient. Thus, in certain forms of the composition the inflammatory disorder is an alimentary tract disorder caused by, or associated with, infection with HIV infection. In addition to HIV, infection with cytomegalovirus can be problematic in AIDS patients.

Bacterial infections are a major cause of morbidity and mortality in many circumstances, but particularly in immunocompromised patients whereby problems are often due to the overgrowth of a normally commensal organism. For example. HIV patients do not possess a fully functional immune system, and so the propensity exists for normally harmless bacteria to reproduce to levels that are damaging to the host. An HIV-infected individual may exhibit gastrointestinal symptoms because the normal balance of intestinal flora and other elements of the nonspecific immune defense system are altered, allowing antigens to cross the gut wall. Such infections typically produce mucosal ulcerations that can result in pain, bleeding, diarrhea, and GI perforation. However, in one form of the method the microbe is a bacterium. In HIV-infected patients, bacteria are sometimes capable of generating inflammatory responses in the alimentary tract. It is thought that infection of cells of the alimentary tract with virus leads to various changes in the wall of the tract. As for viruses, the foreign antigens presented by bacteria can generate strong inflammatory responses in the body leading to various pathologies of the gut and other tissues.

The bacterium may be a commensal bacterium, and may be a Gram negative commensal bacterium. The term “commensal” refers to one of two partners living in permanent close association which gains a benefit from the association without causing serious disadvantage under normal conditions. Commensal bacteria are non-pathogenic bacteria which form part of the normal flora of a healthy human alimentary tract. Examples of commensal Gram negative genera may be selected from the group of genera consisting of Enterobacter, Escherichia, Klebsiella, Bacteroide, Proteus, Salmonella, Serratia, Veillonella, Fusobacteria and Listeria. Inflammation may result from the translocation of microbe or microbial product triggering the pathway across the barrier normally presented by the wall of the alimentary tract. In some circumstances, these organisms grow to levels not normally seen in healthy individuals leading to the presence of large amounts of bacteria or bacterial products in the lumen of the alimentary tract, thereby further exacerbating the inflammatory response.

The microbe may be one that is caused by, or associated with, an opportunistic infection in a subject infected with HIV. For example, opportunistic infections resulting from microbes such as Cryptosporidium spp, Microsporidium spp, Mycobacterium spp (including M. tuberculosis and M. avium), Bartonella spp, Candida spp, Cryptococcus spp, Histoplasma spp, Leishmania spp and Cytomegalovirus are commonly noted in HIV-infected patients.

In light of the above, it will be understood that for the purposes of the present methods, it is unnecessary for the subject to have ingested a foreign organism to produce an opportunistic infection. However in some forms of the method, the opportunistic infection is the result of exposure to a foreign organism.

In one embodiment of the composition, the inflammatory disorder is an immune disorder. For example, HIV preferentially infects activated, memory CD4+ lymphocytes expressing requisite co-receptors. The gut mucosa is the largest immune organ of the body, which in health is characterised by low-level inflammation and constitutive expression of chemokines and cytokines. Upon infection, chemotactic chemokines recruit additional activated immune cells leading to immune disorders such as inflammation of the gut. Alternatively, or additionally, the inflammatory response may be due to an opportunistic infection as described supra. Another example of an immune disorder is that of celiac disease, whereby certain antigenic proteins (typically found in grain) trigger an immune response, leading to inflammation.

In one embodiment of the composition, the immune disorder is a depletion of a T-cell population in the subject. As will be understood, T-cells are involved in cellular immunity not only in the wall of the alimentary tract, but also at many other sites in the body such as the blood, and lymph nodes. T-cells positive for the CD4+ marker are particularly important in HIV infection, given that the virus utilizes these cells for replication, and destroys large numbers in the process. Thus, significant declines in CD4+ T-cell numbers are noted in the blood and/or gut-associated lymphoid tissue of HIV-infected patients. As a result, these patients become severely immunocompromized and often succumb to any one of a number of opportunistic infections characteristic of AIDS.

In one embodiment of the composition, the immune disorder is an immune activation disorder. A unique aspect of HIV infection (among other chronic viral infections) is the chronic activation of the patient's immune system. This chronic activation is often associated with enhanced disease progression in the subject, leading to a more rapid or more complete appearance of AIDS. Immune activation can be measured in the laboratory by reference the expression of markers such as CD69, KI-67, HLA-DR and CD-38, as well as the level of CD4+ T-cells.

It is proposed that the binding of the ligands of the composition to the microbe or microbial product are capable of binding to the products and neutralizing the potentially inflammatory effects.

The ligand may be any pharmaceutically acceptable molecule capable of binding to a microbe or microbial product. Typically, the ligand is a protein molecule (including a glycoprotein molecule), and may be a polypeptide as short as an octamer. The protein ligand may be monomeric, dimeric, trimeric or polymeric.

In one embodiment, the microbial product against which the ligand is directed is DNA, CpG-containing DNA, RNA, flagellin, beta-glucan, peptidoglycan, and lipopeptide.

The term “antibody” as used herein includes both antibodies and antigen binding fragments thereof. Exemplary antibody fragments include, but are not limited to, a single chain antibody, Fab, Fab′ F(ab′)2, Fv or scFv. The preferred anti-LPS antibody is whole antibody in the form of or derived from hyperimmune bovine colostrum.

In one form of the composition the antibody or fragment thereof or derivative thereof is produced by immunization of an animal with a microbe or a microbial product. Polyclonal antibodies capable of binding to a microbe or microbial product may be obtained by the immunization of an animal, and obtaining the antibodies via a bodily fluid, such as blood, a secretion of a gland or cell, egg, milk or colostrum.

Methods for generating hyperimmune sera, milk, colostra and the like are known in the art. However, to the best of the applicant's knowledge the present specification discloses for the first time the generation of hyperimmune materials directed against microbes and microbial products that have the ability to translocate across the lining of the alimentary tract to trigger an inflammatory response.

The method for generating the hymperimmune material may comprise the step of purifying the microbe or the microbial product from other potentially immunogenic molecules. For example, microbes and microbial products can isolated by methods such as high and low speed centrifugation, optionally with the use of gradients formed using sucrose, percoll, cesium and the like. Chromotagraphic methods such as size exclusion chromatography, affinity chromatography, high performance liquid chromatography, reverse phase chromatography, and the like are also useful. Electrophoretic methods (such as capillary electrophoresis), filtration methods (such as tangential flow ultrafiltration), partitioning methods (such as protein precipitation) are further examples of useful methods.

For the production of hyperimmune material, the microbe or microbial product (whether or not purified) is administered to an animal, typically by way of injection (for example, via the IM, subcutanteous, intraperitoneal, or intravenous route). The microbe or microbial product may be combined with an adjuvant to increase the immune response generated by the animal. The skilled person is familiar with many potentially useful adjuvants, such as Freund's complete adjuvant, alum, and squalene.

The animal may be dosed with the microbe or microbial product at intervals over a period of days, weeks or months. At the conclusion of the immunization regime, the hyperimmune material (such as blood, milk or colostrums) is harvested. Antibodies in the hyperimmune material may be harvested by any suitable method, including any by method described supra.

The microbe or microbial product used in vaccination to produce antibodies may be a Gram negative bacterium or may be derived from a Gram negative bacterium. The antigen may comprise the bacterium or bacterial product in any of a range of forms. It may be in the form of whole live, attenuated or killed bacteria or may be in the form at least partly separated from bacterial cell walls.

In one embodiment, the bacterium or bacterial product used for immunization is derived from a commensal Gram negative bacterium selected from the group of genera consisting of Enterobacter, Escherichia, Klebsiella, Bacteroide, Proteus, Salmonella, Serratia, Veillonella and Fusobacteria.

In one embodiment the microbial product is separated from the bacterial cell walls by application of an effective amount of shear, homogenization or heat or by effective combinations thereof.

In one embodiment the composition comprises antibodies from colostrum or a colostrum extract, further characterised in that the colostrum is enriched in anti-microbial or anti-microbial product antibodies when compared with colostrum obtained without vaccination.

In one embodiment of the method the polyclonal antibodies are obtained from a hyperimmune material. The hyperimmune material is enriched when compared with corresponding material in which the animal has not been challenged with the antigen in question.

The animal used to produce the hyperimmune material may be any suitable animal, including a human. However, since human milk may contain potentially transmissible human pathogens, one form of the method provides that the antibody is not human-derived. In any event, animals that produce large quantities of milk are preferred. In this regard, ungulates (and cows in particular), are animals useful for the generation of hyperimmune material.

In one embodiment of the method, the “hyperimmune material” is hyperimmune dairy derived material such as milk particularly colostral milk (colostrum) and the like which is enriched in antibodies or fragments thereof and which is derived from an animal source. The hyperimmune dairy material is preferably hyperimmune colostrum.

In another embodiment the hyperimmune material is derived from bird eggs. A subtype of immunoglobulin known as IgY can be easily extracted from the yolk. Typically, the yolk is first defatted and the IgY isolated by methods identical or similar to those used for skim milk.

The term “colostrum” as used herein includes colostral milk; processed colostral milk such as colostral milk processed to partly or completely remove one or more of fat, cellular debris, lactose and casein; and colostral milk or processed colostral milk which has been dried by for example, freeze drying, spray drying or other methods of drying known in the art. Colostral milk is generally taken from a mammal such as a cow within five days after parturition. Preferably the mammalian colostrum is bovine colostrum retained from the first 4 days post parturition, more preferably bovine colostrum retained from the first 2 days post parturition, even more preferably bovine colostrum retained from the first day post parturition, and most preferably bovine colostrum retained from the first milking post parturition.

Preferably the colostrum collected from the cow comprises at least 4% total protein (weight %), more preferably 5%, more preferably at least 8%, more preferably at least 10%, more preferably at least 20%.

Preferably the ratio of IgG to total protein of the colostrum collected from the cow is at least 10%, more preferably 20%, more preferably at least 30%, more preferably at least 40%, more preferably at least 50%.

It will be understood that in certain embodiments the present compositions are distinguished from the prior art at least in part due to the higher levels of anti-microbe or anti-microbe or microbial product antibodies. For example, studies of dairy products, show low levels of microbe or microbial product antibodies are naturally present in these materials. For example in normal colostrum there are no significant levels of antibody against microbes or microbial products (<100 mg per litre of liquid colostrum of IgG ligand or equivalent molar amount. This corresponds to <1 g per kg of colostrum solids of IgG ligand or equivalent molar amount of other ligand. In certain forms of the method the levels of microbe or microbial product is in excess of those normally found in dairy products.

The hyperimmune dairy material preferably contains at least 3 g per kilogram of product which is IgG directed against the microbe or microbial product, or an equivalent molar concentration of the anti-microbe or microbial product antibody. For example the hyperimmune material may contain at least 5 g, at least 10 g or at least 15 g anti-microbe or anti-microbe or microbial product antibody per kg of hyperimmune material on the basis of the dry weight of components. The upper end of the range of antibody concentration will depend on factors such as the dose, the disease state and the health of the patient. The hyperimmune material may, for example contain no more than 80 g such as no more than 60 g, no more than 50 g or no more than 40 g anti-microbe or anti-microbe or microbial product antibody per kg of hyperimmune material on the basis of the dry weight of components.

In one embodiment of the method the ligand is administered to the subject as a composition. The composition may in one embodiment comprise a carrier admixed with the ligand prior to administration, for example, by mixing a composition of hyperimmune colostrum from immunized cows or one or more processed components thereof with conventional foods and/or pharmaceutically acceptable excipients. The ratio of enriched product relative to conventional dairy material from unvaccinated animals may, for example, be at least 4, such as at least 10 in a comparative ELISA assay.

In another embodiment part or all of the antibodies specific for the microbe or microbe or microbial product are extracted from the colostrum and used to prepare a composition for administration.

In one embodiment the hyperimmune material binds microbe or microbial product taken from at least one Gram negative organism selected from the group of genera consisting of Enterobacter, Escherichia, Klebsiella, Bacteroides, Proteus, Salmonella, Serratia, Veillonella and Fusobacteria.

Preferably the hyperimmune material binds at least two of the above family, more preferably at least 3, even more preferably at least 4.

The degree of enrichment in material selected from antibodies capable of binding to the microbe or microbe or microbial product may be at least 4 times, for example at least 10 times the level found in corresponding unvaccinated animals with respect each of 2 microbe or microbial product molecules, each of 3 microbe or microbial product molecules or each of 4 microbe or microbial product molecules as determined by standard ELISA.

In one embodiment, low molecular weight moieties have been substantially removed from the colostrum or the colostrum extract. By substantially removed is meant that at least 75% and preferably 90% of the low molecular weight moieties are removed.

In a preferred example of this embodiment at least 75% (such as at least 90% or substantially complete removal) of, moieties of molecular weight less than 30 kDa have been removed from the colostrum or the colostrum extract. Preferably molecular weight moieties less than 60 kDa have been substantially removed from the colostrum or colostrum extract.

In one embodiment, the hyperimmune material comprises immunogenic material selected from antibody and antibody fragments which bind microbe or microbial product of commensal bacteria. Preferably the antibody or antibody fragment is a polyclonal antibody or a polyclonal antibody fragment of bovine origin.

The composition may further contain growth factor molecules that are normally found in milk or colostrum. These factors may produce a synergism with the anti-microbe or microbial product antibodies contained in the composition. Exemplary growth factors include TGF-beta-1, TGF-beta-2, IGF-1, IGF-2, EGF, FGF and PDGF.

In one embodiment the antibody or antibody fragment is generated by vaccinating a dairy cow, wherein the vaccine comprises microbial products substantially separated from the wall fragments of the microbe as a result of the application of shear, homogenisation or heat or by effective combinations thereof. The preferred conditions used to effect the separation can be established by carrying out the following test: Centrifuge the whole cell suspension which has been treated to effect the separation and remove the whole cells and substantial cell fragments. Collect the resultant cell-free liquor and run on a gel according to the following protocol: A) Analysis of LPS From Cell Free Liquors Add an equal volume of standard phenol solution to a liquor sample obtained as described previously, vortex and incubate in a waterbath at 65° C. for 15 mins vortexing every 5 mins to denature protein in the liquor. Centrifuge for 10 mins at 4° C. and recover aqueous phase to a fresh tube.

The vaccination regimen leading to the production of hyperimmune colostrum preferably involves the injection of an animal with 0.3 to 15 mL of vaccine on 2 to 8 occasions prior to parturition. The time period between successive vaccinations is 1 to 4 weeks, more preferably 2 to 3 weeks. Methods for production and processing of colostrum are provided in U.S. Pat. No. 5,780,028 the contents of which are incorporated by reference.

The processed hyperimmune colostrum can be formulated as a tablet or as a powder within a capsule or as an additive to a drink mix as described in U.S. Pat. No. 5,780,028, the contents of which is herein incorporated by reference.

Preferably the composition for administration to the patient further comprises a food-grade antimicrobial moiety, such as citrus extracts and iodine based antiseptics. In one preference the antimicrobial moiety is the grapefruit seed extract of the chemical family diphenol hydroxybenzene sold under the product name Citricidal by NutriBiotics of Ripton, Vt., USA.

The composition for administration to the patient may be the hyperimmune material but may and preferably will be derived from the hyperimmune material.

For example, in the case of colostrum the composition for administration to the patient may have been processed using a detailing operation, more preferably using a defatting operation and an operation to remove cellular debris, more preferably a defatting operation, an operation to remove cellular debris and an operation to remove salts, sugars, other low molecular weight entities and some water.

In one embodiment the composition for administration to the patient comprises colostrum components which contain the ligand in dried form. Other components such as selected from the group consisting of adjuvants, carriers, drugs, and other actives may be present in the composition and may be intimately mixed before, during or after the drying process. The composition comprising colostrum may be dried by lyophilisation or other method known in the art for drying colostrum.

In one embodiment the composition for administration to the patient comprises at least three quarters of the lyophilised material by dry weight of the composition based on the dry weight of lypholized hyperimmune colostrum.

Preferably the colostrum collected from the cow comprises at least 4% total protein (weight %), more preferably 5%, more preferably at least 8%, more preferably at least 10%, more preferably at least 20%.

Preferably the ratio of IgG to total protein of the colostrum collected from the cow is at least 10%, more preferably 20%, more preferably at least 30%, more preferably at least 40%, more preferably at least 50%.

The composition for administration to the patient may be in the form of preparations such as food additives, aqueous solutions, oily preparations, emulsions, gels, etc., and these preparations may be administered orally, topically, rectally, nasally, bucally, or vaginally. The preparations may be administered in dosage formulations containing conventional non-toxic acceptable carriers and may also include one or more acceptable additives, including acceptable salts, polymers, solvents, buffers, excipients, bulking agents, diluents, excipients, suspending agents, lubricating agents, adjuvants, vehicles, delivery systems, emulsifiers, disintegrants, absorbents, preservatives, surfactants, colorants, flavorants or sweeteners. A preferred dosage form of the present invention is a powder for incorporation into beverages, pills, syrup, capsules, tablets, granules, beads, chewable lozenges or food additives, using techniques known in the art.

The composition for administration to the patient may, for example, contain additives such as described in our co-pending application WO/2006/053383.

The present methods require the administration of an effective amount of a ligand. As used herein, the term “effective amount” is intended to mean a therapeutically effective amount or a prophylactically effective amount of a ligand of the present invention. Where the method is for prevention, the effective amount does not necessarily provide complete prophylaxis. The subject may still contract an inflammatory disorder or become infected with HIV, however the disorder or infection may be delayed or of a lower severity than would otherwise be noted in the absence of treatment with a subject ligand. Similarly, a therapeutically effective amount does not necessarily result in the subject returning to complete health. As is well understood, HIV integrates into the genome of a cell of the subject, and may never be completely cleared from the body. Similarly, disorders of the alimentary tract may be chronic in nature, persisting until the death of the subject. It is nonetheless proposed that the present methods and compositions will at least improve the health or wellbeing of a subject, without necessarily completely preventing or completely curing disease.

The composition may be administered to the patient in a range of forms depending on the area of the alimentary tract which is subject to the disorder (or is at risk of being subject to the disorder), and condition of the patient. Examples of forms include mouth washes gargles, suppositories, tablets, caplets, pastes, syrups, or in powder or water dispensable powder or granular forms. Where the composition is administered in tablet form the tablet may be made by compressing or moulding the active ingredient, with one or more accessory ingredients optionally included. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made in a suitable machine, by moulding together a mixture of the powdered active ingredient and a suitable carrier, moistened with an inert liquid diluent.

In some embodiments, the methods described herein are for use in connection with subjects having been already infected with HIV, or at risk of infection with HIV. As used herein, the term “infection with HIV” is intended to mean the entry of a virion of HIV-1 or HIV-2 to a cell of the subject leading to the replication of the virion. Given the differences in pathogenicity between the two genotypes, the invention provides greater advantage against infection with HIV-1.

In one form of the method the subject is a human at risk of infection with HIV. The subject may be at risk of horizontal or vertical infection. Horizontal infection may occur due to exposure to body fluids such as blood, semen, vaginal secretions, breast milk, saliva, or exudates from wounds or skin and mucosal lesions, containing free virions or infected cells (both free and cell-associated virions can establish mucosal infection). Transmission is more likely with higher concentrations of virions, which can be very high during primary infection, even if asymptomatic.

Horizontal transmission can occur via the sexual route: homosexual or heterosexual intercourses, including in vitro fertilization. The highest risk of sexual transmission is associated with unprotected anal receptive intercourse. In women, viral invasion occurs mostly through the non-keratinized squamous epithelium of the vagina and ectocervix, as well as through the single-layer columnar epithelium of the endocervix. The endocervical canal is filled with mucus, providing a barrier against the ascent of pathogens. However, ovulation is accompanied by hydration and alkalinization of the mucus plug, possibly decreasing its barrier function. Infection in women can also ensue when HIV invades the single-layer columnar epithelium of the rectum following receptive anal intercourse. In men, viral invasion occurs most frequently through the inner foreskin and the penile urethra as a consequence of penile-vaginal or penile-anal intercourse. Thinly stratified columnar epithelial cells line most of the urethra except for the fossa navicularis near the external meatus, which is covered by non-keratinized squamous epithelium. The glans penis and the outer foreskin are protected by keratinized squamous epithelium, which provides a strong mechanical barrier against HIV invasion. By contrast, a thin and poorly keratinized squamous epithelium covers the inner foreskin, rendering this site vulnerable to HIV invasion.

HIV infection commonly targets the lower gastrointestinal tract as an initial infection site following receptive anal intercourse in humans and direct inoculation in macaques, and as a secondary infection site following rapid dissemination from mucosal foci or acute systemic infection. The rectal mucosa contains simple columnar epithelial cells, and the lamina propria is a rich source of lymphoid cells and lymphoid nodules. The relevant target cells for infection in the lower gastrointestinal tract are likely to be primarily CD4+ memory T cells.

The upper gastrointestinal tract, lined by non-keratinized squamous epithelium in the oropharynx and the oesophagus, and by single-layer columnar epithelium in the stomach and the small intestine, is another site of mucosal HIV invasion. In adults, transmission in the upper gastrointestinal tract occurs following contact with HIV-containing semen during fellatio.

Horizontal infections may also occur via the parenteral route by use of contaminated injection equipment (by drug users, by sportsmen using injectable anabolic steroid, by blood transfusion and blood product recipients, or by haemophiliacs). Risk of transmission by blood transfusion is low, but nevertheless exists. This is because of a window period of about 20 days between infection and seroconversion, as detected by screening methods such as PCR.

The subject may be at risk of vertical infection during pregnancy, during delivery (intra partum), or via breast feeding. Risk of HIV infection from mother-to-child is approximately 25% in European and North American countries, and it is higher in Africa. In infants, HIV invasion in the upper gastrointestinal tract occurs after exposure to or ingestion of infected maternal blood and genital secretions during birth, as well as infected milk during breast feeding.

Thus, in certain embodiments of the method, the subject is at risk for HIV infection. Such subjects include male homosexuals, intravenous drug users, sex workers, blood product recipients, health workers, laboratory workers, and children of HIV-infected mothers.

In one form of the method the subject is already infected with HIV, and optionally under treatment with anti-retroviral agent(s). In another embodiment the subject is infected with HIV and has an existing disorder of the alimentary tract, or is at risk of contracting such a disorder.

The step of administering the ligand may be carried out by any method deemed appropriate by a person skilled in the art. Typically, the method requires that the ligand is applied to a lining of the alimentary tract. This is most readily achieved by oral ingestion of the ligand. However, other means of administration may be effective, such as rectal administration, or by the direct application of the ligand to the desired site. For example, where it is desired to administer the ligand directly to the duodenum, an endoscope may be used for delivery. Where the target site is the colon, colonoscopy may be used.

In terms of dosage, the skilled person will be capable of determining an effect amount of antibody through no more than routine means. Dosage will vary according to variables such as the type of antibody, the size of the subject and the desired clinical endpoint. A physician skilled in the treatment and prevention of HIV will be able to conduct routine studies to identify an effective amount of antibody according to a given clinical scenario. For example, a simple study would include titrating the amount of antibody from a very low level, up to a level where the required clinical endpoint is achieved. Determination that the endpoint is achieved could be via clinical signs and symptoms. Laboratory determinations could also be used to determine the endpoint. In one form of the method, the amount of anti-microbe or microbial product antibody administered may be from about 1, 2, 3, 4, 5, 6, 7, 8, 9, 10, 11, 12, 13, 14, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 500, 600, 700, 800, 900, 1000, 1200, 1400, 1600, 1800, 2000, 2500, 300, 3500, 3600, 4000, 4500, 5000, 5500, 6000, 6500 mg per day.

In one form of the method, the anti-microbe or microbial product antibody is administered at about 3600 mg per day.

In one embodiment, the total daily dosage is administered as two equal dosage forms at an interval of about 12 hours.

In another embodiment the total daily dosage is administered as six equal dosage forms.

In one embodiment of the method, the composition is co-administered with one or more anti-retroviral drugs. It is not necessary for the administration for the ligand and the anti-retroviral drug is performed at the same time. Indeed, the two agents may be administered minutes, hours, days, weeks or even months apart. The anti-retroviral drug(s) may be any one or more of the following agents: Zidovudine (AZT), Abacavir, Emtricitabine (FTC), Lamivudine (3TC), Didanosine (ddI), Stavudine (d4T), Zalcitabine (ddC), Nevirapine, Efavirenz, Delavirdine, Tenofovir, Enfuvirtide (T20), Maraviroc (CCR5), Lopinavir, Atazanavir, Fosamprenvir, Amprenavir, Saquinavir, Indinavir, Nelfinavir, Raltegravir, and Elvitegravir.

In another aspect the present invention provides a method for decreasing the HIV load in a subject, the method comprising the step of administering to a subject in need thereof an effective amount of a ligand capable of binding to a microbe or microbial product molecule. It is proposed that by inhibiting the translocation of microbe or microbial product across a lining of the alimentary tract, inflammation and/or immune activation of the alimentary tract and/or the systemic circulation is also inhibited. This may in turn lead to a decrease in the numbers of infiltrating immune cells thereby decreasing the pool of cells available for HIV replication. With a decrease in the number of permissive host cells, viral load is decreased.

The ligand may be a polypeptide, and in certain embodiments of the method the ligand is an antibody, or a fragment thereof, or a functional equivalent thereof. The antibody may be produced by immunization of an animal with a microbe or microbial product (or an intact or semi-intact microbe containing microbe or microbial product). The antibody, or fragment thereof, or functional equivalent thereof resulting from the immunization may be present in or obtained from a hyperimmune colostrum or milk of the animal. The animal may be a non-human animal, such as an ungulate. In one embodiment of the method, the ungulate is a cow.

The ligand is administered by application to a lining of the alimentary tract, such as that achieved orally or rectally.

In certain embodiments of the method, the subject is a human infected with HIV, or at risk of infection with HIV. Typical subjects for whom the present invention will be advantageous are described supra.

In one form of the method, the subject is under treatment with an antiretroviral agent. In some embodiments, the method comprises co-administration of an antiretroviral agent.

Inhibition of inflammation in the gut can also improve the efficacy of antiretroviral drugs. It has been found that inflammation of the gut interfered with the action of highly active antiretroviral therapy (HAART), allowing a reservoir of HIV to build up in the gut and preventing the virus from being eradicated. Accordingly, the present invention provides a method for improving the efficacy of an antiretroviral agent in the treatment of HIV infection in a subject, the method comprising the step of administering to a subject in need thereof an effective amount of a ligand capable of binding to a microbe or microbial product molecule. In certain embodiments of the method, the subject is a human infected with HIV, or at risk of infection with HIV. Typical subjects for whom the present invention will be advantageous are described supra.

The efficacy of many of the methods of treatment or prevention described herein may be tested by any one or more of the known measures of treatment efficacy, including mean time weighted CD4+ T-cell change over 24 weeks. Secondary endpoints are directed to plasma microbe or microbial product levels, activated CD4+ and CD8+ T-cell levels, mean CD4+ change from baseline, mean CD4+ percentage change from baseline, change in microbial translocation markers: sCD14, 16S RNA fragments, change in activated Cd4+ and CD8+ T-cells from baseline CD38+ HLA-DR+, CD45RO+, change in plasma HIV RNA, change in plasma immune activation markers.

Efficacy may also be tested by visual inspection of the alimentary tract ether via surgical means (open or minimally invasive), gastroscopy, proctoscopy and the like.

The present invention is also predicated at least in part on the proposal that the administration of a ligand capable of binding to bacterial lipopolysaccharide (LPS) is useful in the treatment and/or prevention of inflammatory disorders. Without wishing to be limited by theory, it is thought that the ligand inhibits the translocation of LPS (whether it is free, or as a part of a bacterium) across the barrier normally presented by the lining of the alimentary tract. Inhibition of the translocation of bacteria or LPS may subsequently inhibit inflammation of the alimentary tract, or even in remote tissues of the body. It is proposed that local inflammation of the alimentary tract may be problematic leading to various negative clinical outcomes such as colitis, destruction of CD4+ cells, immune activation, and the like. The treatment or prevention of Inflammation at sites remote to the alimentary tract are included within the scope of this invention. Given that alimentary tract is well vascularized, inflammatory mediators such as interleukin-1, tumor necrosis factor, interleukin-6, nterleukin-11, interleukin-8/chemokines, eotaxin, interleukin-16, interleukin-17, colony stimulating factors, interleukin-3, interleukin-4, interleukin-5, interleukin-7, interleukin-9, interleukin-10, interleukin-13, interleukin-14, transforming growth factor-b, interleukin-2, interleukin-12, interleukin-15, interferons, and IFN-g-inducing factor are able to reach remote areas of the body to trigger inflammation. In particular reticuloendothelial tissues such as spleen, bone marrow, liver, lymph nodes and the thymus may be affected by inflammatory mediators generated by the alimentary tract.

The present invention also provides a method for treating or preventing and inflammatory disorder, the method comprising the step of administering to a subject in need thereof an effective amount of a ligand capable of binding to a LPS molecule.

The ligands of the present methods are in some embodiments directed to LPS. It is proposed that the ligands are capable of binding to the products and neutralizing the potentially inflammatory effects.

The ligand may be any pharmaceutically acceptable molecule capable of binding to an LPS molecule. LPS is a molecule consisting of a lipid and a polysaccharide (carbohydrate) joined by a covalent bond. LPS is a major component, for example, of the outer membrane of Gram-negative bacteria, contributing greatly to the structural integrity of the bacteria, and protecting the membrane from certain kinds of chemical attack. The only Gram-positive bacterium that possesses LPS is Listeria monocytogenes. The term LPS is not intended to be restrictive to mean the entire LPS molecule (i.e. Regions I, IIa, IIb, and III), and includes fragments thereof. Typically, the ligand is a protein molecule (including a glycoprotein molecule), and may be a polypeptide as short as an octamer. The protein ligand may be monomeric, dimeric, trimeric or polymeric.

The term “antibody” as used herein includes both antibodies and antigen binding fragments thereof. Exemplary antibody fragments include, but are not limited to, a single chain antibody, Fab, Fab′ F(ab′)2, Fv or scFv. The preferred anti-LPS antibody is whole antibody in the form of or derived from hyperimmune bovine colostrum.

In one form of the method, the polyclonal anti-LPS antibody is produced by immunization of an animal with a LPS molecule. Polyclonal antibodies capable of binding to LPS may be obtained by the immunization of an animal, and obtaining the antibodies via a bodily fluid, such as blood, a secretion of a gland or cell, egg, milk or colostrum.

The LPS antigen used in vaccination to produce anti-LPS antibodies may be and preferably is derived from Gram negative bacteria. The antigen may comprise LPS in any of a range of forms. It may be in the form of whole live, attenuated or killed bacteria or may be in the form at least partly separated from bacterial cell walls.

In one embodiment, the LPS antigen used for immunization is derived from a commensal Gram negative bacterium selected from the group of genera consisting of Enterobacter, Escherichia, Klebsiella, Bacteroide, Proteus, Salmonella, Serratia, Veillonella and Fusobacteria.

In one embodiment, LPS is separated, at least in part, by one or more of a range of methods using for example heat, detergents, lysis or mechanical means. Methods of separating LPS from cell walls of bacteria are described in our application WO/2004/078209 (with reference to separation of O-antigen) the contents of which are herein incorporated by reference. In particular the preferred method of separating LPS from cell walls is by application of shear. The LPS antigen used in vaccination can be separated from the bacterial cell walls by application of an effective amount of shear, homogenization or heat or by effective combinations thereof.

In one embodiment the method involves the use of colostrum or a colostrum extract, further characterised in that the colostrum is enriched in anti-microbial or anti-LPS antibodies when compared with colostrum obtained without vaccination.

In one embodiment of the method the polyclonal antibodies are obtained from a hyperimmune material. The hyperimmune material is enriched when compared with corresponding material in which the animal has not been challenged with the antigen in question.

Accordingly, in one embodiment, the medicament comprises hyperimmune colostrum raised in bovine mammals by immunization of the bovine mammals with LPS.

The animal used to produce the hyperimmune material may be any suitable animal, including a human. However, since human milk may contain potentially transmissible human pathogens, one form of the method provides that the antibody is not human-derived. In any event, animals that produce large quantities of milk are preferred. In this regard, ungulates (and cows in particular), are animals useful for the generation of hyperimmune material.

In one embodiment of the method, the “hyperimmune material” is hyperimmune dairy derived material such as milk particularly colostral milk (colostrum) and the like which is enriched in antibodies or fragments thereof and which is derived from an animal source. The hyperimmune dairy material is preferably hyperimmune colostrum.

In another embodiment the hyperimmune material is derived from bird eggs. A subtype of immunoglobulin known as IgY can be easily extracted from the yolk. Typically, the yolk is first defatted and the IgY isolated by methods identical or similar to those used for skim milk.

The term “colostrum” as used herein includes colostral milk; processed colostral milk such as colostral milk processed to partly or completely remove one or more of fat, cellular debris, lactose and casein; and colostral milk or processed colostral milk which has been dried by for example, freeze drying, spray drying or other methods of drying known in the art. Colostral milk is generally taken from a mammal such as a cow within five days after parturition. Preferably the mammalian colostrum is bovine colostrum retained from the first 4 days post parturition, more preferably bovine colostrum retained from the first 2 days post parturition, even more preferably bovine colostrum retained from the first day post parturition, and most preferably bovine colostrum retained from the first milking post parturition.

Preferably the colostrum collected from the cow comprises at least 4% total protein (weight %), more preferably 5%, more preferably at least 8%, more preferably at least 10%, more preferably at least 20%.

Preferably the ratio of IgG to total protein of the colostrum collected from the cow is at least 10%, more preferably 20%, more preferably at least 30%, more preferably at least 40%, more preferably at least 50%.

It will be understood that in certain embodiments the present methods are distinguished from the prior art at least in part due to the higher levels of anti-microbe or anti-LPS antibodies used for administration. For example, studies of dairy products, show low levels of LPS antibodies are naturally present in these materials. For example in normal colostrum there are no significant LPS antibodies (<100 mg per litre of liquid colostrum of IgG ligand or equivalent molar amount of other LPS ligand. This corresponds to <1 g per kg of colostrum solids of IgG ligand or equivalent molar amount of other LPS ligand). In certain forms of the method the levels of LPS is in excess of those normally found in dairy products.

The hyperimmune dairy material preferably contains at least 3 g per kilogram of product which is IgG anti-microbe or anti-LPS antibody, or an equivalent molar concentration of other anti-microbe or anti-LPS antibody. For example the hyperimmune material may contain at least 5 g, at least 10 g or at least 15 g anti-microbe or anti-LPS antibody per kg of hyperimmune material on the basis of the dry weight of components. The upper end of the range of antibody concentration will depend on factors such as the dose, the disease state and the health of the patient. The hyperimmune material may, for example contain no more than 80 g such as no more than 60 g, no more than 50 g or no more than 40 g anti-microbe or anti-LPS antibody per kg of hyperimmune material on the basis of the dry weight of components.

In one embodiment of the method the ligand is administered to the subject as a composition. The composition may in one embodiment comprise a carrier admixed with the ligand prior to administration, for example, by mixing a composition of hyperimmune colostrum from immunized cows or one or more processed components thereof with conventional foods and/or pharmaceutically acceptable excipients. The ratio of enriched product relative to conventional dairy material from unvaccinated animals may, for example, be at least 4, such as at least 10 in a comparative ELISA assay.

In another embodiment part or all of the antibodies specific for the microbe or LPS are extracted from the colostrum and used to prepare a composition for administration.

In one embodiment the hyperimmune material binds LPS taken from at least one Gram negative organism selected from the group of genera consisting of Enterobacter, Escherichia, Klebsiella, Bacteroides, Proteus, Salmonella, Serratia, Veillonella and Fusobacteria. Preferably the hyperimmune material binds at least two of the above family, more preferably at least 3, even more preferably at least 4.

The degree of enrichment in material selected from antibodies capable of binding to the microbe or LPS may be at least 4 times, for example at least 10 times the level found in corresponding unvaccinated animals with respect each of 2 LPS molecules, each of 3 LPS molecules or each of 4 LPS molecules as determined by standard ELISA.

In one embodiment, low molecular weight moieties have been substantially removed from the colostrum or the colostrum extract. By substantially removed is meant that at least 75% and preferably 90% of the low molecular weight moieties are removed.

In a preferred example of this embodiment at least 75% (such as at least 90% or substantially complete removal) of, moieties of molecular weight less than 30 kDa have been removed from the colostrum or the colostrum extract. Preferably molecular weight moieties less than 60 kDa have been substantially removed from the colostrum or colostrum extract.

In one embodiment, the hyperimmune material comprises immunogenic material selected from antibody and antibody fragments which bind LPS of commensal bacteria. Preferably the antibody or antibody fragment is a polyclonal antibody or a polyclonal antibody fragment of bovine origin.

The composition may further contain growth factor molecules that are normally found in milk or colostrum. These factors may produce a synergism with the anti-LPS antibodies contained in the composition. Exemplary growth factors include TGF-beta-1, TGF-beta-2, IGF-1, IGF-2, EGF, FGF and PDGF.

In one embodiment the antibody or antibody fragment is generated by vaccinating a dairy cow, wherein the vaccine comprises LPS substantially separated from the wall fragments of the microbe as a result of the application of shear. This process is described in our copending application PCT/AU2004/00027, which is incorporated herein by reference.

The vaccination regimen leading to the production of hyperimmune colostrum preferably involves the injection of an animal with 0.3 to 15 mL of vaccine on 2 to 8 occasions prior to parturition. The time period between successive vaccinations is 1 to 4 weeks, more preferably 2 to 3 weeks. Methods for production and processing of colostrum are provided in U.S. Pat. No. 5,780,028 the contents of which are incorporated by reference.

The processed hyperimmune colostrum can be formulated as a tablet or as a powder within a capsule or as an additive to a drink mix as described in U.S. Pat. No. 5,780,028.

Preferably the composition for administration to the patient further comprises a food-grade antimicrobial moiety, such as citrus extracts and iodine based antiseptics. In one preference the antimicrobial moiety is the grapefruit seed extract of the chemical family diphenol hydroxybenzene sold under the product name Citricidal by NutriBiotics of Ripton, Vt., USA.

The composition for administration to the patient may be the hyperimmune material but may and preferably will be derived from the hyperimmune material.

For example, in the case of colostrum the composition for administration to the patient may have been processed using a detailing operation, more preferably using a defatting operation and an operation to remove cellular debris, more preferably a defatting operation, an operation to remove cellular debris and an operation to remove salts, sugars, other low molecular weight entities and some water.

In one embodiment the composition for administration to the patient comprises colostrum components which contain the ligand in dried form.

Other components such as selected from the group consisting of adjuvants, carriers, drugs, and other actives may be present in the composition and may be intimately mixed before, during or after the drying process. The composition comprising colostrum may be dried by lyophilisation or other method known in the art for drying colostrum.

In one embodiment the composition for administration to the patient comprises at least three quarters of the lyophilised material by dry weight of the composition based on the dry weight of lypholized hyperimmune colostrum.

Preferably the colostrum collected from the cow comprises at least 4% total protein (weight %), more preferably 5%, more preferably at least 8%, more preferably at least 10%, more preferably at least 20%.

Preferably the ratio of IgG to total protein of the colostrum collected from the cow is at least 10%, more preferably 20%, more preferably at least 30%, more preferably at least 40%, more preferably at least 50%.

The composition for administration to the patient may be in the form of preparations such as food additives, aqueous solutions, oily preparations, emulsions, gels, etc., and these preparations may be administered orally, topically, rectally, nasally, bucally, or vaginally. The preparations may be administered in dosage formulations containing conventional non-toxic acceptable carriers and may also include one or more acceptable additives, including acceptable salts, polymers, solvents, buffers, excipients, bulking agents, diluents, excipients, suspending agents, lubricating agents, adjuvants, vehicles, delivery systems, emulsifiers, disintegrants, absorbents, preservatives, surfactants, colorants, flavorants or sweeteners. A preferred dosage form of the present invention is a powder for incorporation into beverages, pills, syrup, capsules, tablets, granules, beads, chewable lozenges or food additives, using techniques known in the art.

The composition for administration to the patient may, for example, contain additives such as described in our co-pending application WO/2006/053383 the contents of which are herein incorporated by reference.

The present methods require the administration of an effective amount of a ligand. As used herein, the term “effective amount” is intended to mean a therapeutically effective amount or a prophylactically effective amount of a ligand of the present invention. Where the method is for prevention, the effective amount does not necessarily provide complete prophylaxis. The subject may still contract an inflammatory disorder or become infected with HIV, however the disorder or infection may be delayed or of a lower severity than would otherwise be noted in the absence of treatment with a subject ligand. Similarly, a therapeutically effective amount does not necessarily result in the subject returning to complete health. As is well understood, HIV integrates into the genome of a cell of the subject, and may never be completely cleared from the body. Similarly, disorders of the alimentary tract may be chronic in nature, persisting until the death of the subject. It is nonetheless proposed that the present methods and compositions will at least improve the health or wellbeing of a subject, without necessarily completely preventing or completely curing disease.

The terms “suppression” and “suppress” as used herein, refer to the result of administration of composition described herein initiated prior to the onset of clinical signs of an inflammatory gastrointestinal disorder so as to reduce the clinical symptoms or severity of an inflammatory gastrointestinal disorder. The suppression may, but need not be absolute.

The term “treatment” refers to administration initiated after the onset of clinical signs of an inflammatory gastrointestinal disorder so as to reduce or eliminate the clinical signs of an inflammatory gastrointestinal disorder. Treatment may or may not be absolute.

The composition may be administered to the patient in a range of forms depending on the area of the alimentary tract which is subject to the disorder (or is at risk of being subject to the disorder), and condition of the patient. Examples of forms include mouth washes gargles, suppositories, tablets, caplets, pastes, syrups, or in powder or water dispensable powder or granular forms. Where the composition is administered in tablet form the tablet may be made by compressing or moulding the active ingredient, with one or more accessory ingredients optionally included. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made in a suitable machine, by moulding together a mixture of the powdered active ingredient and a suitable carrier, moistened with an inert liquid diluent.

In some embodiments, the methods described herein are for use in connection with subjects having been already infected with HIV, or at risk of infection with HIV. As used herein, the term “infection with HIV” is intended to mean the entry of a virion of HIV-1 or HIV-2 to a cell of the subject leading to the replication of the virion. Given the differences in pathogenicity between the two genotypes, the invention provides greater advantage against infection with HIV-1.

In one form of the method the subject is a human at risk of infection with HIV. The subject may be at risk of horizontal or vertical infection. Horizontal infection may occur due to exposure to body fluids such as blood, semen, vaginal secretions, breast milk, saliva, or exudates from wounds or skin and mucosal lesions, containing free virions or infected cells (both free and cell-associated virions can establish mucosal infection). Transmission is more likely with higher concentrations of virions, which can be very high during primary infection, even if asymptomatic.

Horizontal transmission can occur via the sexual route: homosexual or heterosexual intercourses, including in vitro fertilization. The highest risk of sexual transmission is associated with unprotected anal receptive intercourse. In women, viral invasion occurs mostly through the non-keratinized squamous epithelium of the vagina and ectocervix, as well as through the single-layer columnar epithelium of the endocervix. The endocervical canal is filled with mucus, providing a barrier against the ascent of pathogens. However, ovulation is accompanied by hydration and alkalinization of the mucus plug, possibly decreasing its barrier function. Infection in women can also ensue when HIV invades the single-layer columnar epithelium of the rectum following receptive anal intercourse. In men, viral invasion occurs most frequently through the inner foreskin and the penile urethra as a consequence of penile-vaginal or penile-anal intercourse. Thinly stratified columnar epithelial cells line most of the urethra except for the fossa navicularis near the external meatus, which is covered by non-keratinized squamous epithelium. The glans penis and the outer foreskin are protected by keratinized squamous epithelium, which provides a strong mechanical barrier against HIV invasion. By contrast, a thin and poorly keratinized squamous epithelium covers the inner foreskin, rendering this site vulnerable to HIV invasion.

HIV infection commonly targets the lower gastrointestinal tract as an initial infection site following receptive anal intercourse in humans and direct inoculation in macaques, and as a secondary infection site following rapid dissemination from mucosal foci or acute systemic infection. The rectal mucosa contains simple columnar epithelial cells, and the lamina propria is a rich source of lymphoid cells and lymphoid nodules. The relevant target cells for infection in the lower gastrointestinal tract are likely to be primarily CD4+ memory T cells.

The upper gastrointestinal tract, lined by non-keratinized squamous epithelium in the oropharynx and the oesophagus, and by single-layer columnar epithelium in the stomach and the small intestine, is another site of mucosal HIV invasion. In adults, transmission in the upper gastrointestinal tract occurs following contact with HIV-containing semen during fellatio.

Horizontal infections may also occur via the parenteral route by use of contamined injection equipment (by drug users, by sportsmen using injectable anabolic steroid, by blood transfusion and blood product recipients, or by haemophiliacs). Risk of transmission by blood transfusion is low, but nevertheless exists. This is because of a window period of about 20 days between infection and seroconversion, as detected by screening methods such as PCR.

The subject may be at risk of vertical infection during pregnancy, during delivery (intra partum), or via breast feeding. Risk of HIV infection from mother-to-child is approximately 25% in European and North American countries, and it is higher in Africa. In infants, HIV invasion in the upper gastrointestinal tract occurs after exposure to or ingestion of infected maternal blood and genital secretions during birth, as well as infected milk during breast feeding.

Thus, in certain embodiments of the method, the subject is at risk for HIV infection. Such subjects include male homosexuals, intravenous drug users, sex workers, blood product recipients, health workers, laboratory workers, and children of HIV-infected mothers.

In one form of the method the subject is already infected with HIV, and optionally under treatment with anti-retroviral agent(s). In another embodiment the subject is infected with HIV and has an existing disorder of the alimentary tract, or is at risk of contracting such a disorder.

The step of administering the ligand may be carried out by any method deemed appropriate by a person skilled in the art. Typically, the method requires that the ligand is applied to a lining of the alimentary tract. This is most readily achieved by oral ingestion of the ligand. However, other means of administration may be effective, such as rectal administration, or by the direct application of the ligand to the desired site. For example, where it is desired to administer the ligand directly to the duodenum, an endoscope may be used for delivery. Where the target site is the colon, colonoscopy may be used.

In terms of dosage, the skilled person will be capable of determining an effect amount of antibody through no more than routine means. Dosage will vary according to variables such as the type of antibody, the size of the subject and the desired clinical endpoint. A physician skilled in the treatment and prevention of HIV will be able to conduct routine studies to identify an effective amount of antibody according to a given clinical scenario. For example, a simple study would include titrating the amount of antibody from a very low level, up to a level where the required clinical endpoint is achieved. Determination that the endpoint is achieved could be via clinical signs and symptoms. Laboratory determinations could also be used to determine the endpoint.

The anti-LPS antibody may be administered in a dose in the range of from 1.05 to 260 mg per day. In another embodiment, the anti-LPS antibody may be administered in a solid oral unit dosage form comprising in the range from 1.05 to 260 mg polyclonal anti-LPS antibody. The oral solid dose form may comprise at least 20% by weight hyperimmune bovine colostrum wherein solid bovine colostrum comprises at least 7% by dry weight of the powder of IgG.

In one embodiment, the oral dose form may be administered at a dose of about 5 mg to about 25000 mg per day, 10 mg to about 20000 mg per day, 25 mg to about 15000 mg per day, or 100 mg to about 10000 mg per day.

In another embodiment, the oral dose form may be administered at a dose of about 150 mg to about 6500 mg per day.

In one embodiment the antibodies are present in the composition for oral administration in an amount sufficient to provide from at least about 7% by dry weight of the composition of IgG. In another embodiment the antibodies are present in the composition for oral administration in an amount sufficient to provide from at least about 50% by weight of the composition of IgG.

Accordingly, the oral dose form may comprise 10.5 mg to 3250 mg IgG, e.g. 10, 20, 40, 60, 80, 100, 120, 140, 160, 180, 200, 250, 300, 350, 400, 450, 500, 550, 600, 650, 700, 750, 800, 850, 900, 950, 1000, 1100, 1250, 1500, 1750, 2000, 2200, 2400, 2600, 2800, 300, 3200 or 3250 mg IgG.

In one embodiment antibodies specific to the antigen (e.g. anti-LPS antibodies) are present in the composition for oral administration in an amount sufficient to provide from about 10% specific IgG of the weight of IgG.

Accordingly, the oral dose form may comprise 1.05 mg to 325 mg anti-LPS IgG, e.g. 1, 2, 4, 6, 8, 10, 12, 14, 16, 18, 20, 25, 30, 35, 40 45, 50, 55, 60, 65, 70, 75, 80, 85, 90, 95, 100, 110, 125, 150, 175, 200, 220, 240, 260, 280, 300, 320, or 325 mg anti-LPS IgG.

In another embodiment, the oral dose form may be administered at a dose 3600 mg per day. Accordingly, the oral dose form may comprise about 252 mg to about 1800 mg IgG, or 25.2 to about 180 mg anti-LPS IgG.

In one embodiment, the anti-LPS antibody is administered for about 4 weeks.

In another aspect, the present invention provides a medicament for treatment or suppression of inflammatory gastrointestinal disease in a human subject having HIV infection comprising an effective amount of a polyclonal anti-LPS antibody.

In another aspect, the present invention provides the use of a polyclonal anti-LPS antibody in manufacture of a medicament for administration to a human subject for treatment or suppression of HIV mediated inflammatory bowel disease.

In one embodiment, the medicament is a solid oral unit dosage form comprising in the range from 1.05 to 325 mg polyclonal anti-LPS antibody.

In another aspect, the present invention provides a solid oral unit dose form for treatment or suppression of inflammatory bowel disease in a patient suffering from HIV infection, the solid dosage form comprising at least 20% by weight of hyperimmune bovine colostrum powder based on the total weight of oral dosage form, said hyperimmune bovine colostrum powder comprising at least 7% by dry weight of IgG.

In one embodiment the unit dosage form comprises in the range from 1.05 to 325 mg polyclonal anti-LPS antibody.

In another embodiment, the anti-LPS antibody may be administered concomitantly with antiretroviral drugs. It is not necessary for the administration for the ligand and the anti-retroviral drug is performed at the same time. Indeed, the two agents may be administered minutes, hours, days, weeks or even months apart. The anti-retroviral drug(s) may be any one or more of the following agents: Zidovudine (AZT), Abacavir, Emtricitabine (FTC), Lamivudine (3TC), Didanosine (ddI), Stavudine (d4T), Zalcitabine (ddC), Nevirapine, Efavirenz, Delavirdine, Tenofovir, Enfuvirtide (T20), Maraviroc (CCR5), Lopinavir, Atazanavir, Fosamprenvir, Amprenavir, Saquinavir, Indinavir, Nelfinavir, Raltegravir, and Elvitegravir.

In another aspect the present invention provides a method for decreasing the HIV load in a subject, the method comprising the step of administering to a subject in need thereof an effective amount of a ligand capable of binding to a LPS molecule. It is proposed that by inhibiting the translocation of LPS across a lining of the alimentary tract, inflammation and/or immune activation of the alimentary tract and/or the systemic circulation is also inhibited. This may in turn lead to a decrease in the numbers of infiltrating immune cells thereby decreasing the pool of cells available for HIV replication. With a decrease in the number of permissive host cells, viral load is decreased.

The ligand may be a polypeptide, and in certain embodiments of the method the ligand is an antibody, or a fragment thereof, or a functional equivalent thereof. The antibody may be produced by immunization of an animal with a LPS (or an intact or semi-intact microbe containing LPS). The antibody, or fragment thereof, or functional equivalent thereof resulting from the immunization may be present in or obtained from a hyperimmune colostrum or milk of the animal. The animal may be a non-human animal, such as an ungulate. In one embodiment of the method, the ungulate is a cow.

The anti-LPS antibody may be prepared by immunizing a mammal with LPS from multiple E. coli strains. The mammal or avian may be immunized with LPS selected from the group consisting of O6, O8, O15, O25, O27, O63, O78, O114, O115, O128, O148, O153, O159, and other LPS associated with enterotoxigenic E. coli.

The mammal may be immunized with LPS selected from the group consisting of O78, O6, O8, O129 and O153 LPS. The LPS may comprise O78 LPS.

Accordingly, the medicament comprising hyperimmune colostrum may be raised in bovine mammals by immunization of the bovine mammals with LPS from two or more strains of bacteria.

Methods of preparing LPS/O antigen are known in the art and described in WO/2004/078209, which is incorporated herein by reference. Methods of preparing hyperimmune bovine colostrum (HIBC) are also described in WO/2004/078209.

The ligand is administered by application to a lining of the alimentary tract, such as that achieved orally or rectally.

In certain embodiments of the method, the subject is a human infected with HIV, or at risk of infection with HIV. Typical subjects for whom the present invention will be advantageous are male homosexuals, intravenous drug users, sex workers, blood product recipients, health workers, laboratory workers, and children of HIV-infected mothers.

In one form of the method, the subject is under treatment with an antiretroviral agent. In some embodiments, the method comprises co-administration of an antiretroviral agent.

Inhibition of inflammation in the gut can also improve the efficacy of antiretroviral drugs. It has been found that inflammation of the gut interfered with the action of highly active antiretroviral therapy (HAART), allowing a reservoir of HIV to build up in the gut and preventing the virus from being eradicated. Accordingly, the present invention provides a method for improving the efficacy of an antiretroviral agent in the treatment of HIV infection in a subject, the method comprising the step of administering to a subject in need thereof an effective amount of a ligand capable of binding to a LPS molecule. In certain embodiments of the method, the subject is a human infected with HIV, or at risk of infection with HIV. Typical subjects for whom the present invention will be advantageous are male homosexuals, intravenous drug users, sex workers, blood product recipients, health workers, laboratory workers, and children of HIV-infected mothers.

The efficacy of many of the methods of treatment or prevention described herein may be tested by any one or more of the known measures of treatment efficacy, including mean time weighted CD4+ T-cell change over 24 weeks. Secondary endpoints are directed to plasma lipopolysaccharide levels, activated CD4+ and CD8+ T-cell levels, mean CD4+ change from baseline, mean CD4+ percentage change from baseline, change in microbial translocation markers: sCD14, 16S RNA fragments, change in activated Cd4+ and CD8+ T-cells from baseline CD38+ HLA-DR+, CD45RO+, change in plasma HIV RNA, change in plasma immune activation markers.

The methods and compositions of the present invention may result in an increase in CD4+ T-cells, of about 1, 2, 3, 5, 10, 15, 20, 25, 30, 35, 40, 45, 50, 55, 60, 65, 70, 75, 80, 85, 90, 95 and over 99% relative to untreated control, or levels prior to the treatment.

The methods and compositions of the present invention may result in an increase in CD4+ T-cells, of about 1, 2, 3, 5, 10, 15, 20, 25, 30, 35, 40, 45, 50, 55, 60, 65, 70, 75, 80, 85, 90, 95 and over 99% relative to untreated control, or levels prior to the treatment.

The methods and compositions of the present invention may result in a decrease in serum LPS of about 1, 2, 3, 5, 10, 15, 20, 25, 30, 35, 40, 45, 50, 55, 60, 65, 70, 75, 80, 85, 90, 95 and over 99% relative to untreated control, or levels prior to the treatment.

In one embodiment, the methods and compositions of the present invention may result in a decrease in serum LPS of about 10 pg/mL. In another embodiment, the methods and compositions of the present invention may result in a decrease in serum LPS of about 15 pg/mL. In another embodiment, the methods and compositions of the present invention may result in a decrease in serum LPS of about 20 pg/mL. In another embodiment, the methods and compositions of the present invention may result in a decrease in serum LPS of about 25 pg/mL.

The methods and compositions of the present invention may result in a decrease in soluble CD14 (sCD14), of about 1, 2, 3, 5, 10, 15, 20, 25, 30, 35, 40, 45, 50, 55, 60, 65, 70, 75, 80, 85, 90, 95 and over 99% relative to untreated control, or levels prior to the treatment.

In one embodiment, the methods and compositions of the present invention may result in a decrease in sCD14 of about 70 ng/mL. In another embodiment, the methods and compositions of the present invention may result in a decrease in sCD14 of about 70 ng/mL. In another embodiment, the methods and compositions of the present invention may result in a decrease in sCD14 of about 140 ng/mL. In another embodiment, the methods and compositions of the present invention may result in a decrease in sCD14 of about 210 ng/mL. In another embodiment, the methods and compositions of the present invention may result in a decrease in sCD14 of about 280 ng/mL. In another embodiment, the methods and compositions of the present invention may result in a decrease in sCD14 of about 500 ng/mL.

T cell (e.g. CD4+ T-cell) responses may be quantified using methods known in the art, for example ELISPOT assays, flow cytometry or useful immunodetection methods described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed T regulatory cells. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP)

Efficacy may also be tested by visual inspection of the alimentary tract ether via surgical means (open or minimally invasive), gastroscopy, proctoscopy and the like.

In yet a further aspect the present invention provides a composition comprising a ligand capable of binding to a LPS molecule, the ligand present in an amount such that upon administration to a subject having an inflammatory disorder, the disorder is ameliorated. In one form of the composition the inflammatory disorder is a disorder of the alimentary tract such as ulcerative colitis, Crohn's disease, irritable bowel syndrome, celiac disease. In another form the inflammatory disorder is caused by, or associated with infection of the alimentary tract with a microbe such as a rotavirus, norovirus, adenovirus, sapovirus, astrovirus, hepatitis A virus, hepatitis B virus, hepatitis C virus, hepatitis delta agent, hepatitis E virus or hepatitis G virus, HIV, cytomegalovirus, Enterobacter spp, Escherichia spp, Klebsiella spp, Bacteroides spp, Proteus spp, Salmonella spp, Serratia spp, Veillonella spp Fusobacteria spp, Listeria spp, Cryptosporidium spp, Microsporidium spp, Mycobacterium spp, Bartonella spp, Candida spp, Cryptococcus spp, Histoplasma spp, Leishmania spp.

The ligand may be a polypeptide, and in certain embodiments of the method the ligand is an antibody, or a fragment thereof, or a functional equivalent thereof. The antibody may be produced by immunization of an animal with a LPS molecule. The antibody, or fragment thereof, or functional equivalent thereof resulting from the immunization may be present in or obtained from a hyperimmune colostrum or milk of the animal. The animal may be a non-human animal, such as an ungulate. In one embodiment of the method, the ungulate is a cow.

Where the composition is a liquid composition, the amount of ligand present may be from about 1, 2, 3, 4, 5, 6, 7, 8, 9, 10, 11, 12, 13, 14, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 or 400 mg per mL.

Where the composition is in solid form, the amount of ligand present may be from about 0.1, 2, 3, 4, 5, 6, 7, 8, 9, 10, 11, 12, 13, 14, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 or 400 mg per gram

The present invention will now be more fully described by reference to the following non-limiting Examples

### EXAMPLES

### Example 1

### Method for Confirming that Hyperimmune Sample Material Comprising Anti-Microbial Product Antibody Binds with a Commensal Bacterial Microbial Product

1. Procurement of a commensal Gram negative strain. The following strains were obtained from the University of Melbourne: Enterobacter aerogenes, Klebsiella pneumoniae, Pseudomonas aeruginosa and Salmonella typhimurium.
2. Culturing the strain. Enterobacter aerogenes was cultured on horse blood agar (HBA) plates in 37° C. incubator for 16 hours, Klebsiella pneumoniae was cultured on Luria agar (LA) plates in 37° C. incubator for 16 hours, Pseudomonas aeruginosa was cultured on horse blood agar (HBA) plates in 37° C. incubator for 16 hours and Salmonella typhimurium was cultured on Luria agar (LA) plates in 37° C. incubator for 16 hours.
3. Purifying microbial product from the culture. The procedure was based on Hitchcock, P. J. & Brown, T. M. (1983). Morphological heterogeneity among Salmonella chemotypes in silver stained polyacrylamide gels. J. Bacteriol. 154, 269-277, with modifications as follows:
Bacteria are collected from the plate the following day using a sterile cotton swab and suspended in phosphate buffered saline (PBS), to an optical density of 2.0 measured at 600 nm.
1.0 ml of the suspension is transferred to microcentrifuge tube, centrifuged at 10 000 g for 3 minutes at room temperature. The supernatant is discarded.
Resuspend the bacterial pellet in 200 μl of lysis buffer (1M Tris-HCl (pH 6.8), 2% SDS, dH2O) and boil sample for 10 minutes.
Add 5 μl of proteinase K (stock 20 mg/ml), vortex and incubate at 60° C. for 60 minutes.

Add 300 μl of phenol to the sample and incubate at 65° C. for 15 minutes. Vortex every 5 minutes.

Centrifuge samples at 10 000 g for 10 minutes at 4° C.
Remove clear aqueous phase (top phase) to a clean tube. Add equal amount of chloroform to remove any residual phenol in the sample.
Centrifuge samples at 10 000 g for 5 minutes at 4° C.
Remove top phase to a clean tube, store samples at −20° C. until needed.
   4. Electrophoresis of the purified microbial product on SDS PAGE. The procedure is as follows
Prepare a 15% acrylamide solution for resolving gel and a 4% acrylamide solution for separating gel.
Load equal volumes for all the samples into a total volume (in loading dye: 62.5 mM Tris-HCl pH 6.8, 10% glycerol, 2% SDS, 40 mM DTT, 0.125 5 bromophenol blue, dH2O) of no greater than 15 μl per lane.
Load the samples into the wells and run them at 50 V until the dye has reached the resolving gel, then switch to 130 V and run the samples until the dye has left the gel. (Running buffer: 3.03 g Tris, 14.4 g Glycine, 1.0 g SDS, 1 L dH₂O).
   5. Transfer of the microbe or microbial product onto a PVDF membrane followed by a Western blot using a solution of the hyperimmune sample material—a replicate using non-hyperimmune material is also prepared. The procedure used was as follows:
After running samples on Tris-Tricine-SDS-PAGE transfer the bands onto polyvinylidene difluorite (PVDF) membrane for 2.5 hours at 100 V at 4° C. in transfer buffer pH 8.3 (25 mM Tris, 192 mM Tricine, 10% v/v MeOH, dH2O).
Block membranes overnight at 4° C. in PBS-Tween 0.1%+5% skim milk powder.
Rinse blots with PBS/T 0.1% for 5 minutes, shaking.
Incubate blots with hyperimmune material diluted 1/200 in PBS/T+5% skim milk powder (SMP) or non-hyperimmune material for 2 hours with gentle shaking.
Rinse blots and wash with PBS/T, two times for 5 minutes each wash.
Incubate blots with goat □ bovine IgG-HRP 1/20 000 in PBS/T+5% SMP for 1 hour, with gentle shaking.
Rinse and wash blots as above.
Drain excess PBS/T and place damp blots on an overhead sheet. Prepare ECL reagent; drop 1 ml on each membrane and leave for 1 minute. Place another overhead sheet on top of membrane and blot excess ECL reagent ensuring where the film is placed is dry.
Expose blots to film for required time (1-3 minutes) and develop film.

### Example 2

### Production of Hyperimmune Colostrum Containing Polyclonal Anti-Microbial Product Antibodies

Step 1—Production of Vaccine for Dairy Cattle

The procedures for preparing microbe or microbial product-containing antigen reported in Pub. No. WO/2004/078209 International Application No. PCT/AU2004/000277 are used.
Step 2—The procedures for preparing anti-microbe or microbial product antibodies from vaccinated cattle reported in Pub. No. WO/2004/078209 International Application No. PCT/AU2004/000277 are used.

### Example 3

### Detection of Flagellin and Presence of Flagellin Antibodies in a Bovine Colostrum Composition

### Materials & Methods

### Bacterial Strains

Human enterotoxigenic Escherichia coli strains B7A O148:H28, H10407 O78:H11, E123-7 O128:H21, B2C O6:H16, E11881A O25:H24, E8772/0 O153:H12, human adherent invasive E. coli strain LF82 O83:H1, bovine ETEC strain K99, human isolate E. coli strain HS and E. coli lab strain HB101 were used.

### Growth Conditions

All bacterial strains were passaged three times on 0.35% Luria Bertani (LB) swarm agar, grown at 30° C. Bacteria that grew at the outermost edge of swarm were then used as a starter culture for 10 ml LB broths (HB101 showed non-motile phenotype). Broths were grown as a static culture, overnight, at 30° C. 10 μl of overnight culture was used to inoculate a fresh 100 ml LB broth, which was grown overnight at 30° C. Bacterial motility was checked by wet mount hanging drop, by light microscopy.

### Purification of Flagellin

1. Overnight cultures (100 ml) were centrifuged at 3 800 rpm for 30 min at 4° C. to pellet bacteria. Bacterial pellets were resuspended in a total volume of 1 ml of 150 mM NaCl: 10 mM HCl (pH ˜1.5) to dissociate flagella from the bacterial surface.
2. The bacterial suspension was transferred to a 2 ml Eppendorf tube and placed on a rotary wheel at top speed, incubated for 1 hour at RT ° C. This suspension was centrifuged at 8 000×g for 15 mins at 4° C.
3. 600 μl of each supernatant was neutralised with the addition of 50 μl of 50 mM Tris: 10 mM NaOH. The neutralised solutions were placed into a 1.5 ml Beckman ultra-centrifuge tube.
4. The supernatants were ultra-centrifuged at 100 000×g for 90 mins at 4° C. Supernatants were carefully removed and discarded after centrifugation; pellets were resuspended in a volume of 100 μl of PBS, overnight at 4° C. Flagellin preparations were stored at −20° C. before being used.

### SDS-PAGE & Western Blot

Flagellin samples were run on 10% SDS-PAGE (Laemmli buffer system) before being Coomassie stained or transferred to PVDF membrane for Western blot. Western blot was performed using Travelan® batch TRV001 at a concentration of 5 mg/ml in PBS. A secondary goat α bovine IgG-HRP conjugate (Sigma) was used at a concentration of 1/20 000 before the blot was developed using ECL western blotting substrate.

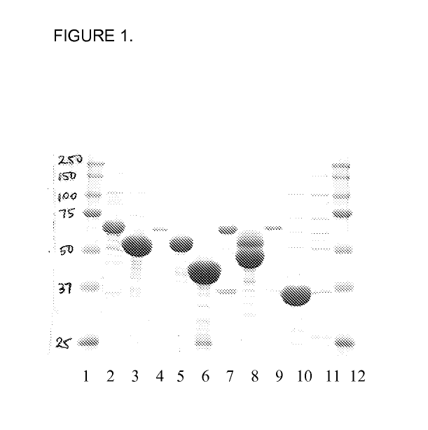

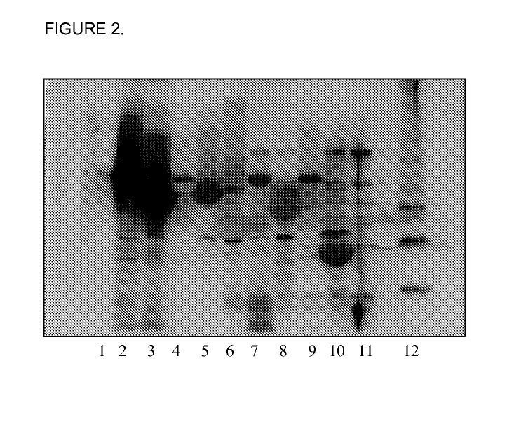

The results of this study are shown herein as FIG. 1 and FIG. 2.

### Example 4

### Formulation of Bovine Colostrum as Colostrum Powder Tablets

Tablets are formulated according to standard methods based on the following table of ingredients. “HIC Colostrum” is hyperimmune colostrum containing antibodies against flagellin. The total antibody amount per tablet is 600 mg, with the amount of anti-microbe or microbial product antibody about 60 mg.

| | | | | | | |
|---|---|---|---|---|---|---|
| | | Input | | | | |
| RM Code | Raw Material Name | (in mg) | Claim per Tablet | Overage % | UOM | Std |

Actives

| | | | | | | |
|---|---|---|---|---|---|---|
| RM5238P | HIC Colostrum | 600.0 | 600 mg | 0% | mg | Anadis |

Excipients

| | | | | | | |
|---|---|---|---|---|---|---|
| RM087P | Crospovidone XL | 125.0 | | | mg | USP |
| RM5180P | Croscarmellose Sodium | 115.0 | | | mg | BP |
| RM5181P | Hydrogenated Vegetable Oil | 25.0 | | | mg | BP |
| RM004P | Colloidal Silica Anhydrous | 25.0 | | | mg | BP |
| RM5145P | Calcium Carbonate 100 Mesh | 60.0 | | | mg | BP |
| RM5441P | Calcium Carbonate (DC Grade) | 90.0 | | | mg | BP |
| | Equiv. Corn Starch | | | (2.7 mg) | | |
| RM080P | Calcium Hydrogen Phosphate | 200.0 | | | mg | BP |
| RM053P | Microcystalline Cellulose | 200.0 | | | mg | BP |
| RM086P | Talc | 7.5 | | | mg | BP |
| RM053P | Magnesium Stearate | 7.5 | | | mg | BP |
| | Total | 1455.0 | mg | | | |

### Example 5

### Co-Administration of HIV-Infected Subject with Raltegravir and Bovine Colostrum Powder Tablet

A 35 year old male AIDS patient with documented HIV-1 infection is administered Raltegravir 400 mg twice daily, in addition bovine colostrum twice daily. The colostrum is provided in a tabletized form of a powder, as described in Example 4, and is administered as 2×400 m capsules twice daily.

Finally, it is understood that various other modifications and/or alterations may be made without departing from the spirit of the present invention as outlined herein.

Future patent applications may be filed on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or redefine the invention or inventions.

### Example 6

### Method for Confirming that Hyperimmune Sample Material Comprising Anti-LPS Antibody Binds with LPS Derived from Commensal Bacteria

1. Procurement of a commensal Gram negative strain. The following strains were obtained from the University of Melbourne: Enterobacter aerogenes, Klebsiella pneumoniae, Pseudomonas aeruginosa and Salmonella typhimurium.
2. Culturing the strain. Enterobacter aerogenes was cultured on horse blood agar (HBA) plates in 37° C. incubator for 16 hours, Klebsiella pneumoniae was cultured on Luria agar (LA) plates in37° C. incubator for 16 hours, Pseudomonas aeruginosa was cultured on horse blood agar (HBA) plates in 37° C. incubator for 16 hours and Salmonella typhimurium was cultured on Luria agar (LA) plates in 37° C. incubator for 16 hours.
3. Purifying LPS from the culture. The procedure was based on Hitchcock, P. J. & Brown, T. M. (1983). Morphological heterogeneity among Salmonella lipopolysaccharide chemotypes in silver stained polyacrylamide gels. J. Bacteriol. 154, 269-277, with modifications as follows:
Bacteria are collected from the plate the following day using a sterile cotton swab and suspended in phosphate buffered saline (PBS), to an optical density of 2.0 measured at 600 nm.
1.0 ml of the suspension is transferred to microcentrifuge tube, centrifuged at 10 000 g for 3 minutes at room temperature. The supernatant is discarded.
Resuspend the bacterial pellet in 200 μl of lysis buffer (1M Tris-HCl (pH 6.8), 2% SDS, dH2O) and boil sample for 10 minutes.
Add 5 μl of proteinase K (stock 20 mg/ml), vortex and incubate at 60° C. for 60 minutes.
Add 300 μl of phenol to the sample and incubate at 65° C. for 15 minutes. Vortex every 5 minutes.
Centrifuge samples at 10 000 g for 10 minutes at 4° C.
Remove clear aqueous phase (top phase) to a clean tube. Add equal amount of chloroform to remove any residual phenol in the sample.
Centrifuge samples at 10 000 g for 5 minutes at 4° C.
Remove top phase to a clean tube, store samples at −20° C. until needed.
   4. Electrophoresis of the purified LPS on SDS PAGE. The procedure was as follows
Prepare a 15% acrylamide solution for resolving gel and a 4% acrylamide solution for separating gel.
Load equal volumes for all the samples into a total volume (in loading dye: 62.5 mM Tris-HCl pH 6.8, 10% glycerol, 2% SDS, 40 mM DTT, 0.125 5 bromophenol blue, dH2O) of no greater than 15 μl per lane.
Load the samples into the wells and run them at 50 V until the dye has reached the resolving gel, then switch to 130 V and run the samples until the dye has left the gel. (Running buffer: 3.03 g Tris, 14.4 g Glycine, 1.0 g SDS, 1 L dH₂O).
   5. Transfer of the LPS onto a PVDF membrane followed by a Western blot using a solution of the hyperimmune sample material—a replicate using non-hyperimmune material is also prepared. The procedure used was as follows:
After running samples on Tris-Tricine-SDS-PAGE transfer the bands onto polyvinylidene difluorite (PVDF) membrane for 2.5 hours at 100 V at 4° C. in transfer buffer pH 8.3 (25 mM Tris, 192 mM Tricine, 10% v/v MeOH, dH2O).
Block membranes overnight at 4° C. in PBS-Tween 0.1%+5% skim milk powder.
Rinse blots with PBS/T 0.1% for 5 minutes, shaking.
Incubate blots with hyperimmune material diluted 1/200 in PBS/T+5% skim milk powder (SMP) or non-hyperimmune material for 2 hours with gentle shaking.
Rinse blots and wash with PBS/T, two times for 5 minutes each wash.
Incubate blots with goat □ bovine IgG-HRP 1/20 000 in PBS/T+5% SMP for 1 hour, with gentle shaking.
Rinse and wash blots as above.
Drain excess PBS/T and place damp blots on an overhead sheet. Prepare ECL reagent; drop 1 ml on each membrane and leave for 1 minute. Place another overhead sheet on top of membrane and blot excess ECL reagent ensuring where the film is placed is dry.
Expose blots to film for required time (1-3 minutes) and develop film.

### Example 7

### Production of Hyperimmune Colostrum Containing Polyclonal Anti-LPS Antibodies

### Step 1—Production of Vaccine for Dairy Cattle

The procedures for preparing LPS-containing antigen reported in Pub. No. WO/2004/078209 International Application No. PCT/AU2004/000277 (the contents of which are herein incorporated by reference) were used.
Step 2—The procedures for preparing anti-LPS antibodies from vaccinated cattle reported in Pub. No. WO/2004/078209 International Application No. PCT/AU2004/000277 (the contents of which are herein incorporated by reference) were used.

### Example 8

### Formulation of Bovine as Colostrum Powder Tablets

Tablets were formulated according to standard methods based on the following table of ingredients. “HIC Colostrum” is hyperimmune colostrum containing antibodies against LPS. The total antibody amount per tablet is 600 mg, with the amount of anti-LPS antibody about 60 mg.

| | | | | | | |
|---|---|---|---|---|---|---|
| | | Input | | | | |
| RM Code | Raw Material Name | (in mg) | Claim per Tablet | Overage % | UOM | Std |

Actives

| | | | | | | |
|---|---|---|---|---|---|---|
| RM5238P | HIC Colostrum | 600.0 | 600 mg | 0% | mg | Anadis |

Excipients

| | | | | | | |
|---|---|---|---|---|---|---|
| RM087P | Crospovidone XL | 125.0 | | | mg | USP |
| RM5180P | Croscarmellose Sodium | 115.0 | | | mg | BP |
| RM5181P | Hydrogenated Vegetable Oil | 25.0 | | | mg | BP |
| RM004P | Colloidal Silica Anhydrous | 25.0 | | | mg | BP |
| RM5145P | Calcium Carbonate 100 Mesh | 60.0 | | | mg | BP |
| RM5441P | Calcium Carbonate (DC Grade) | 90.0 | | | mg | BP |
| | Equiv. Corn Starch | | | (2.7 mg) | | |
| RM080P | Calcium Hydrogen Phosphate | 200.0 | | | mg | BP |
| RM053P | Microcystalline Cellulose | 200.0 | | | mg | BP |
| RM086P | Talc | 7.5 | | | mg | BP |
| RM053P | Magnesium Stearate | 7.5 | | | mg | BP |
| | Total | 1455.0 | mg | | | |

### Example 9

### Co-Administration of HIV-Infected Subject with Raltegravir and Bovine Colostrum Powder Tablet

A 35 year old male AIDS patient with documented HIV-1 infection is administered Raltegravir 400 mg twice daily, in addition bovine colostrum as described herein twice daily. The colostrum is provided in a tabletized form of a powder, as described in Example 1, and is administered as 2×400 m capsules twice daily.

Finally, it is understood that various other modifications and/or alterations may be made without departing from the spirit of the present invention as outlined herein.

### Example 10

### Reduction in the Levels of Intestinal Microbial Products in HIV Infected Persons

### Study Objectives

To evaluate the effect of bovine colostrum ingestion on immune activation in HIV-infected persons. Specifically, the effect of ingestion of BCP on translocation of bacterial LPS and other microbial products across the intestinal mucosa into the bloodstream, and the proportions of T cells expressing an activated phenotype were evaluated.

### Study Design

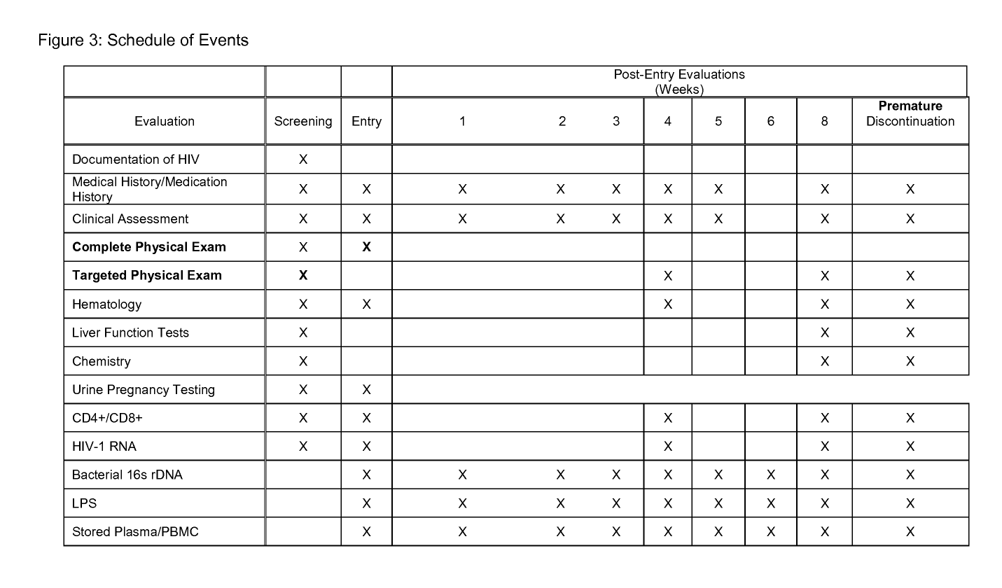

A single-arm, open label, before-and-after exploratory trial was performed to evaluate the effect of 2 weeks of BCP administration on plasma levels of intestinal microbial products and the frequency of activated T cells in chronically HIV-infected, untreated human volunteers not receiving antiretroviral therapy. During the study period, subjects underwent periodical clinical evaluations and blood draws (see schedule of events in FIG. 3).

### Rationale for a Before-and-after Study Design

The before-and-after approach enhances the efficiency of the study by allowing each subject to serve as his/her own control; this reduces inter-individual variability and maximizes the opportunity to detect small treatment-induced differences in this patient population which will expectedly be difficult to recruit. The endpoints in this study are suitable for this type of design, because they were expected to change relatively rapidly in response to the study intervention, and be rapidly reversible, as shown by the effect of antibiotic therapy in laboratory animals in which levels of microbial products are measured before and after the therapeutic intervention. Moreover, because the expected within-subject variation of the study endpoints over time were incompletely understood at trial commencement, this design strengthens the conclusions of the study by providing both evidence of a change in study endpoints after taking into account the observed variability before the study treatment, and by demonstrating a return to the baseline conditions after the interruption of study treatment.

### Study Treatment

Subjects were all monitored for 2 weeks, prior to treatment. After which the study regimen was initiated, consisting of 600 mg BCP tablets given orally six times a day for 4 weeks, followed by 4 weeks off study treatment.

BCP was prepared as described in Example 7. Each BCP tablet is an uncoated 600 mg oral tablet, which contains 600 mg of freeze-dried BCP, in combination with excipients. Each BCP tablet comprises at least about 7% to at least about 7% by dry weight of the composition of IgG (e.g. at least about 42 mg to at least about 240 mg IgG). Furthermore, each BCP tablet comprises at least about 10% specific IgG of the weight of IgG (e.g. at least 4.2 mg to at least about 24 mg anti-LPS IgG).

The freeze-dried bovine colostrum powder (BCP) is milked from commercial dairy cowherds. As described subra, the cows in these herds, as well as being vaccinated for routine cattle pathogens, have been vaccinated with a vaccine against the outer cell wall antigens of multiple strains of E. coli bacteria, a major organism in human gut microflora. The BCP used is a high-protein (>80%), lactose- and fat-reduced natural product derived from the first milking of commercial dairy cows collected after calving. It is presented before tableting as a concentrated, freeze dried powder. BCP contains approx. 40% antibodies (immunoglobulins) in the dry powder. The main classes of immunoglobulins found in bovine colostrum are IgG (mainly IgG1) and IgA with small amounts of IgM and IgE. The immunoglobulins in BCP have high binding activity against the Lipopolysaccharide (LPS) of Gram-negative bacteria.

### Population

HIV-infected men and women, ≧18 years of age, not receiving antiretroviral therapy at the time of enrollment and for at least the previous 6 months, and without an indication to initiate therapy in the next 3 months at enrollment, with a plasma HIV RNA level ≧1,000 copies/mL and a CD4+ T cell count ≧500 cells/mm³.

### Clinical and Laboratory Evaluations

Laboratory assays were (at baseline and each subsequent time point, unless otherwise noted):
1. Serum urine pregnancy test, as appropriate (baseline only)
2. Complete blood count
3. HIV viral load
4. CD4/CD8 T cell counts
5. Immune activation markers, including CD38, HLA-DR by flow cytometry
6. Lipopolysaccharide by limulus amoebocyte lysate assay
7. Bacterial 16S rDNA levels by DNA extraction and polymerase chain reaction
8. PBMC for cryopreservation

### Statistical Considerations

The primary objective was tested by evaluating the effect of administration of BCP on levels of plasma microbial products in the study population after a 2-week administration period using the Wilcoxon signed-rank test to compare the average of the pre-treatment values to the values observed at the end of the 2-week treatment period and the value one week after discontinuation of therapy. Secondarily, the baseline value (defined as the average of 3 observations prior to study treatment) was compared to each of the on-treatment measurements and each of the post-treatment observations, and the values at the end of the treatment period to the post-treatment values. A similar approach was followed to evaluate the decrease in immune activation after the administration of study treatment. To examine the association between on-treatment levels of plasma levels of microbial products and levels of cellular immune activation, the data was examined graphically, and a repeated-measures regression model fitted using the frequency of activated T cells as the dependent variable and both the level of microbial products in plasma and the study phase (pre-, on- or post-treatment) as the explanatory variables. This allowed both an estimate of the treatment effect on immune activation to be determined and whether the effect is entirely dependent on the intermediary effect on levels of microbial products.

### Results

At weeks 0/1/2/4/6/8, CD4/CD8 counts were measured and the activation markers HLA-DR and CD38 on CD4 and CD8 T cells by flow cytometry; plasma LPS by limulus lysate assay; and soluble CD14 by ELISA.

A subset of 9 subjects (7 males; mean age 40 years) is presented; Data are described as means±SD. Exploratory p-values ≦0.1 are indicated by asterisks. Baseline LPS and sCD14 levels were 49±41 pg/mL and 2220±360 ng/mL, respectively. From baseline to weeks 2 and 4 of BCP administration, LPS levels decreased by 21±43 and 21±15* pg/mL, as did sCD14 levels, by 70±288 and 519±288 ng/mL. From the beginning of BCP to weeks 2 and 4 on BCP, LPS decreased by 15±93 and 15±95 pg/mL, as did sCD14, by 377±278* and 446±408* ng/mL. Changes in sCD14 correlated directly with several indices of immune activation at the end of the dosing period (e.g., correlation coefficients between sCD14 decrease from baseline to week 6 to % CD38+CD4+ T cells; CD38 MFI on CD4+ T cells; % CD38+CD8+ T cells; CD38 MFI on CD8+ T cells at week 6=0.81*; 0.8*; 0.79* and 0.82*.)

Thus, hyperimmune BCP reduced plasma levels of microbial products in HIV-infected persons, and reduced immune activation.

Future patent applications may be filed on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or redefine the invention or inventions.

1. A method for treating or suppressing an inflammatory gastrointestinal disorder in a human subject comprising administering to the subject an effective amount of a medicament comprising a polyclonal anti-LPS antibody.

2. A method according to claim 1, wherein the human subject has a HIV infection.

3. A method according to claim 2, wherein the inflammatory gastrointestinal disorder is HIV mediated inflammatory bowel disorder.

4. A method according to claim 2, wherein the human subject is suffering from AIDS.

5. A method according to claim 1, wherein the medicament comprises hyperimmune colostrum raised in bovine mammals by immunization of the bovine mammals with LPS.

6. A method according to claim 5, wherein the anti-LPS antibody is administered in a dose in the range of from 1.05 to 325 mg per day.

7. A method according to claim 1, wherein the anti-LPS antibody is administered in a solid oral unit dosage form comprising in the range from 1.05 to 325 mg polyclonal anti-LPS antibody.

8. A method according to claim 7, wherein the oral solid dose form comprises at least 20% by weight hyperimmune bovine colostrum wherein solid bovine colostrum comprises at least 7% by dry weight of the powder of IgG

9. A method according to claim 1, wherein medicament comprising hyperimmune colostrum is raised in bovine mammals by immunization of the bovine mammals with LPS from two or more strains of bacteria.

10. A method according to claim 1, the anti-LPS antibody is administered concomitantly with antiretroviral drugs, preferably selected from the group consisting of Zidovudine (AZT), Abacavir, Emtricitabine (FTC), Lamivudine (3TC), Didanosine (ddI), Stavudine (d4T), Zalcitabine (ddC), Nevirapine, Efavirenz, Delavirdine, Tenofovir, Enfuvirtide (T20), Maraviroc (CCR5), Lopinavir, Atazanavir, Fosamprenvir, Amprenavir, Saquinavir, Indinavir, Nelfinavir, Raltegravir, and Elvitegravir.

11. A medicament for treatment or suppression of inflammatory gastrointestinal disease in a human subject having HIV infection comprising an effective amount of a polyclonal anti-LPS antibody.

12. Method of using a polyclonal anti-LPS antibody in manufacture of a medicament for administration to a human subject for treatment or suppression of HIV mediated inflammatory bowel disease.

13. The method according to claim 12, wherein the medicament is a solid oral unit dosage form comprising in the range from 1.05 to 325 mg polyclonal anti-LPS antibody.

14. A solid oral unit dose form for treatment or suppression of inflammatory bowel disease in a patient suffering from HIV infection, the solid dosage form comprising at least 20% by weight of hyperimmune bovine colostrum powder based on the total weight of oral dosage form, said hyperimmune bovine colostrum powder comprising at least 7% by dry weight of IgG.

15. A solid oral unit dosage form according to claim 14, wherein the unit dosage form comprises in the range from 1.05 to 325 mg polyclonal anti-LPS antibody.
